(19) United States
(12) Patent Application Publication
(10) Pub. No.: US 2023/0212239 A1
(43) Pub. Date: Jul. 6, 2023

(54) INHIBITORS OF CBL AUTOINHIBITION AND RELATED METHODS

(71) Applicant: THE HOSPITAL FOR SICK CHILDREN, Toronto (CA)

(72) Inventors: C. Jane MCGLADE, Toronto (CA); Leanne WYBENGA-GROOT, Toronto (CA)

(21) Appl. No.: 18/000,713

(22) PCT Filed: Jun. 4, 2021
(86) PCT No.: PCT/CA2021/050774

§ 371 Date: Dec. 5, 2022

Related U.S. Application Data
(60) Provisional application No. 63/034,767, filed on Jun. 4, 2020.

(51) Int. Cl. C07K 14/47; A61K 31/422; A61K 31/4245; A61K 31/454; A61K 31/7105; A61K 31/711

(52) U.S. Cl. CPC C07K 14/4703; A61K 31/422; A61K 31/454; A61K 31/711; A61K 31/4245; A61K 31/7105; C07K 2319/00

(57) ABSTRACT

Described herein are agents that inhibit CBL autoinhibition, agents that activate CBL, SLAP and/or SLAP2 mimetics, and recombinant SLAP and/or SLAP2 or variants and/or fragments thereof that inhibit CBL autoinhibition. Also described are fusion proteins comprising these molecules as well as methods of inhibiting CBL autoinhibition and related uses thereof.

### FIELD

The present invention relates to CBL. In particular, the present invention relates to agents that interact with CBL and related compositions and methods.

### BACKGROUND

Cbl (named after Casitas B-lineage Lymphoma) is a mammalian gene encoding the protein CBL which is an E3 ubiquitin-protein ligase involved in cell signalling and protein ubiquitination. Mutations to this gene have been implicated in a number of human cancers, particularly acute myeloid leukemia.

U.S. Pat. No. 7,507,801 relates to the identification and cloning of a novel gene, MARS (Modulator of Antigen Receptor Signaling), which is a putative tumor suppressor gene. This patent also relates to the relation of the MARS gene to human cancers and its use in the diagnosis and prognosis of human cancer. This patent also relates to the therapy of human cancers which have a mutation in the MARS gene, or lack the MARS gene, including gene therapy, protein replacement therapy and protein mimetics. Finally, this patent relates to the screening of drugs for cancer therapy.

International Patent Application Publication No. WO 1999/067380 describes that introduction of a vector achieving overexpression of the proto-oncogene c-Cbl in transformed cells, particularly cells transformed by an oncogenic protein tyrosine kinase, reverses the transformed phenotype. This application describes a method for suppression of neoplastic transformation.

U.S. Pat. Application Publication No. 2014/0335106 describes a method of treating cancer by inhibiting expression of ubiquitin associated and SH3 domain containing B (UBASH3B) gene or by inhibiting the activity of UBASH3B protein or a functional variant thereof.

A need exists for the development of a product, composition and/or method that provides the public with a useful alternative.

### SUMMARY OF THE INVENTION

In accordance with an aspect, there is provided an agent that inhibits CBL autoinhibition.

In an aspect, the agent acts independently of phosphorylation.

In an aspect, the agent interacts with a tyrosine-kinase binding domain (TKBD), a linker helix region (LHR), or a RING domain of CBL.

In an aspect, the agent does not bind to the phospho-tyrosine binding site of the TKBD.

In an aspect, the agent interacts with a region of CBL that is distinct from the phospho-tyrosine binding site of the TKBD.

In an aspect, the agent interacts with the CBL regulatory cleft.

In an aspect, the CBL regulatory cleft is framed by the 4H bundle, the EF-hand, and the SH2 domain.

In an aspect, the CBL regulatory cleft is framed by helices αC and αD of the 4H bundle, helix αE2 and loop αE2-αF2 of the EF-hand, and helix αN, loop αN-βA, and strand βA of the SH2 domain.

In an aspect, the agent is a SLAP and/or SLAP2 mimetic.

In an aspect, the agent comprises or consists of a peptide, a polynucleotide, a small molecule, a lipid, a carbohydrate, or a combination thereof.

In an aspect, the peptide is an antibody or fragment thereof, a linear, cyclic, or branched peptide or a combination thereof, a glycopeptide, a fusion peptide, a stapled peptide, a peptidomimetic, or a combination thereof.

In an aspect, the peptide is linked to a small molecule, such as a drug, imaging, or targeting agent. In an aspect, the polynucleotide comprises DNA and/or RNA.

In an aspect, the small molecule is a macrocyclic compound.

In an aspect, the agent is N-[(2-chloro-6-fluorophenyl)methyl]-8-methyl-3,4-dihydro-2H-1,5-benzodioxepine-7-carboxamide; N-(3-bromophenyl)-5-(5-cyclobutyl-1,3,4-oxadiazol-2-yl)thiophene-2-sulfonamide; 3-chloro-N-{3-cyclobutyl-[1,2,4]triazolo[4,3-a]pyridin-8-yl}-4-methoxybenzene-1-sulfonamide; N-[(4-methoxyphenyl)methyl]-3-methyl-1-[3-(trifluoromethyl)benzenesulfonyl]piperidine-3-carboxamide; N-[4-chloro-3-(trifluoromethyl)phenyl]-4-(5-cyclobutyl-1,2,4-oxadiazol-3-yl)thiophene-2-sulfonamide; 5-(2-cyclobutyl-1,3-oxazol-5-yl)-N-[4-(trifluoromethoxy)phenyl]thiophene-2-sulfonamide; 1-(4-methylbenzenesulfonyl)-N-(naphthalen-1-yl)-5-oxopyrrolidine-2-carboxamide; 2-{4-[(4-methylphenyl)methyl]-2,3-dioxopiperazin-1-yl}-N-[4-(propan-2-yl)phenyl]acetamide; N-(2-{4-[4-(5-cyclobutyl-1,2,4-oxadiazol-3-yl)phenyl]piperazin-1-yl}-2-oxoethyl)furan-2-carboxamide; and 5-oxo-3-phenyl-N-[3-(propan-2-yloxy)propyl]-5H-[1,3]thiazolo[3,2-a]pyrimidine-6-carboxamide; or any combination thereof.

In an aspect, the agent is provided in a delivery system, such as a gene therapy platform, a liposome, a nanoparticle, a therapeutic cell treatment, or a combination thereof.

In an aspect, the agent is for treatment of a disease or condition in which CBL inhibition or downregulation is implicated.

In an aspect, the disease or condition in which CBL inhibition or downregulation is implicated is cancer, moyamoya angiopathy, or Noonan syndrome.

In an aspect, the cancer is leukemia, lung cancer, or head and neck cancer.

In an aspect, the leukemia is AML, JMML, CMML, or CML.

In an aspect, the cancer is a cancer in which tyrosine kinase activity is implicated.

In accordance with an aspect, there is provided a SLAP and/or SLAP2 mimetic.

In an aspect, the mimetic comprises or consists of a peptide, a polynucleotide, a small molecule, a lipid, a carbohydrate, or a combination thereof.

In an aspect, the peptide is an antibody or fragment thereof, a linear, cyclic, or branched peptide or a combination thereof, a glycopeptide, a fusion peptide, a stapled peptide, a peptidomimetic, or a combination thereof.

In an aspect, the peptide is linked to a small molecule, such as a drug, imaging, or targeting agent. In an aspect, the polynucleotide comprises DNA and/or RNA.

In an aspect, the small molecule is a macrocyclic compound.

In an aspect, the mimetic is N-[(2-chloro-6-fluorophenyl)methyl]-8-methyl-3,4-dihydro-2H-1,5-benzodioxepine-7-carboxamide; N-(3-bromophenyl)-5-(5-cyclobutyl-1,3,4-oxadiazol-2-yl)thiophene-2-sulfonamide; 3-chloro-N-{3-cyclobutyl-[1,2,4]triazolo[4,3-a]pyridin-8-yl}-4-methoxybenzene-1-sulfonamide; N-[(4-methoxyphenyl)methyl]-3-methyl-1-[3-(trifluoromethyl)benzenesulfonyl]piperidine-3-carboxamide; N-[4-chloro-3-(trifluoromethyl)phenyl]-4-(5-cyclobutyl-1,2,4-oxadiazol-3-yl)thiophene-2-sulfonamide; 5-(2-cyclobutyl-1,3-oxazol-5-yl)-N-[4-(trifluoromethoxy)phenyl]thiophene-2-sulfonamide; 1-(4-methylbenzenesulfonyl)-N-(naphthalen-1-yl)-5-oxopyrrolidine-2-carboxamide; 2-{4-[(4-methylphenyl)methyl]-2,3-dioxopiperazin-1-yl}-N-[4-(propan-2-yl)phenyl]acetamide; N-(2-{4-[4-(5-cyclobutyl-1,2,4-oxadiazol-3-yl)phenyl]piperazin-1-yl}-2-oxoethyl)furan-2-carboxamide; and 5-oxo-3-phenyl-N-[3-(propan-2-yloxy)propyl]-5H-[1,3]thiazolo[3,2-a]pyrimidine-6-carboxamide; or any combination thereof.

In an aspect, the mimetic is provided in a delivery system, such as a gene therapy platform, a liposome, a nanoparticle, a therapeutic cell treatment, or a combination thereof.

In an aspect, the mimetic is for treatment of a disease or condition in which CBL inhibition or downregulation is implicated.

In an aspect, the disease or condition in which CBL inhibition or downregulation is implicated is cancer, moyamoya angiopath, or Noonan syndrome.

In an aspect, the cancer is leukemia, lung cancer, or head and neck cancer.

In an aspect, the leukemia is AML, JMML, CMML, or CML.

In an aspect, the cancer is a cancer in which tyrosine kinase activity is implicated.

In accordance with an aspect, there is provided a recombinant SLAP and/or SLAP2 or a variant and/or fragment thereof that inhibits CBL autoinhibition.

In an aspect, the variant comprises at least about 70%, 75%, 80%, 85%, 90%, 95%, 96%, 97%, 98%, 99%, or 99.5% sequence identity to:

MGNSMKSTPAPAERPLPNPEGLDSDFLAVLSDYPSPDISPPIFRRGEKLR
VISDEGGWWKAISLSTGRESYIPGICVARVYHGWLFEGLGRDKAEELLQL
PDTKVGSFMIRESETKKGFYSLSVRHRQVKHYRIFRLPNNWYYISPRLTF
QCLEDLVNHYSEVADGLCCVLTTPCLTQSTAAPAVRASSSPVTLRQKTVD
WRRVSRLQEDPEGTENPLGVDESLFSYGLRESIASYLSLTSEDNTSFDRK
KKSISLMYGGSKRKSSFFSSPPYFED (SLAP;SEQ ID NO:1)

and/or

MGSLPSRRKSLPSPSLSSSVQGQGPVTMEAERSKATAVALGSFPAGGPAE
LSLRLGEPLTIVSEDGDWWTVLSEVSGREYNIPSVHVAKVSHGWLYEGLS
REKAEELLLLPGNPGGAFLIRESQTRRGSYSLSVRLSRPASWDRIRHYRI
HCLDNGWLYISPRLTFPSLQALVDHYSELADDICCLLKEPCVLQRAGPLP
GKDIPLPVTVQRTPLNWKELDSSLLFSEAATGEESLLSEGLRESLSFYIS
LNDEAVSLDDA (SLAP2; SEQ ID NO:2).

In an aspect, the fragment comprises from about 3 to about 275 (SLAP) or 260 (SLAP2) amino acids, such as from about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 180, about 185, about 190, about 195, about 200, about 205, about 210, about 215, about 220, about 225, about 230, about 235, about 240, about 245, about 250, about 255, about 260, about 265, or about 270 amino acids to about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 180, about 185, about 190, about 195, about 200, about 205, about 210, about 215, about 220, about 225, about 230, about 235, about 240, about 245, about 250, about 255, about 260, about 265, about 270, or about 275 amino acids.

In an aspect, the fragment comprises at least about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 180, about 185, about 190, about 195, about 200, about 205, about 210, about 215, about 220, about 225, about 230, about 235, about 240, about 245, about 250, about 255, about 260, about 265, or about 270 amino acids.

In an aspect, the recombinant SLAP and/or SLAP2 or the active variant and/or fragment thereof is in the form of an antibody or fragment thereof, a linear, cyclic, or branched peptide or a combination thereof, a glycopeptide, a fusion peptide, a stapled peptide, a peptidomimetic, or a combination thereof.

In an aspect, the peptide is linked to a small molecule, such as a drug, imaging, or targeting agent.

In an aspect, the recombinant SLAP and/or SLAP2 or the active variant and/or fragment thereof is for treatment of a disease or condition in which CBL inhibition or downregulation is implicated.

In an aspect, the disease or condition in which CBL inhibition or downregulation is implicated is cancer, moyamoya angiopath, or Noonan syndrome.

In an aspect, the cancer is leukemia, lung cancer, or head and neck cancer.

In an aspect, the leukemia is AML, JMML, CMML, or CML.

In an aspect, the cancer is a cancer in which tyrosine kinase activity is implicated.

In accordance with an aspect, there is provided a fusion protein comprising:
the agent described herein, wherein the agent is a peptide;
the mimetic described herein, wherein the mimetic is a peptide; or
the recombinant SLAP and/or SLAP2 or the active variant and/or fragment thereof described herein; fused to a second peptide.

In an aspect, the second peptide is a therapeutic peptide, an imaging peptide, a targeting peptide, or a combination thereof.

In accordance with an aspect, there is provided a method of inhibiting CBL autoinhibition, the method comprising administering to a subject in need thereof the agent described herein; the mimetic described herein; the recombinant SLAP and/or SLAP2 or the active variant and/or fragment thereof described herein; or the fusion protein described herein.

In an aspect, the method is for treating a disease or condition in which CBL inhibition or downregulation is implicated.

In an aspect, the disease or condition in which CBL inhibition or downregulation is implicated is cancer, moyamoya angiopath, or Noonan syndrome.

In an aspect, the cancer is leukemia, lung cancer, or head and neck cancer.

In an aspect, the leukemia is AML, JMML, CMML, or CML.

In an aspect, the cancer is a cancer in which tyrosine kinase activity is implicated.

In accordance with an aspect, there is provided a use of the agent described herein; the mimetic described herein; the recombinant SLAP and/or SLAP2 or the active variant and/or fragment thereof described herein; or the fusion protein described herein for inhibiting CBL autoinhibition.

In an aspect, the use is for treatment of a disease or condition in which CBL inhibition or downregulation is implicated.

In an aspect, the disease or condition in which CBL inhibition or downregulation is implicated is cancer, moyamoya angiopath, or Noonan syndrome.

In an aspect, the cancer is leukemia, lung cancer, or head and neck cancer.

In an aspect, the leukemia is AML, JMML, CMML, or CML.

In an aspect, the cancer is a cancer in which tyrosine kinase activity is implicated.

In accordance with an aspect, there is provided a method of screening for an agent that inhibits CBL autoinhibition and/or an agent that is a SLAP and/or SLAP2 mimetic, the method comprising applying the agent to a composition comprising CBL and detecting a change in CBL activation, wherein an increase in CBL activation suggests that the agent inhibits CBL autoinhibition and/or is a SLAP and/or SLAP2 mimetic.

In an aspect, CBL activation is determined by measuring assembly of polyubiquitin chains by CBL. In an aspect, the method further comprises validating the agent.

In an aspect, validating the agent comprises detecting ubiquitination in an immunoassay, such as an immunoblot, following application of the agent to CBL.

In an aspect, the CBL comprises the TKBD-LHR-RING region.

In an aspect, the CBL is recombinant.

In an aspect, the method is a high-throughput method.

In accordance with an aspect, there is provided an agent identified by the method described herein.

The novel features of the present invention will become apparent to those of skill in the art upon examination of the following detailed description of the invention. It should be understood, however, that the detailed description of the invention and the specific examples presented, while indicating certain aspects of the present invention, are provided for illustration purposes only because various changes and modifications within the spirit and scope of the invention will become apparent to those of skill in the art from the detailed description of the invention and claims that follow.

### BRIEF DESCRIPTION OF THE DRAWINGS

The present invention will be further understood from the following description with reference to the Figures, as follows:

FIG. 1: CBL interacts with C-terminal tail of SLAP2. A) Cartoon of CBL and SLAP2 domains, with arrow indicating interaction between TKBD and SLAP2 C-tail region. B) SDS-PAGE gel stained with Coomassie blue for CBL and SLAP2 proteins co-purified under low salt conditions from Duet-His-Δlinker-hSLAP2-CBL (261), its truncated version (254), Duet-His-hSLAP2-CBL 29-261 Δ198-229, and its truncated version 29-254 Δ198-229. C) Sequence alignment of the C-tail region of SLAP proteins, with residue numbering based on human SLAP2. Conserved Tyr248 is marked with a red box. Secondary structure prediction for mSLAP2 (Jnet pred) and confidence score (Jnet Rel) as calculated by Jnet is shown, with e, -, and h representing β-strand, coil and α-helix, respectively. The secondary structure elements of mSLAP2 as determined by x-ray crystallography are shown in green, with the line and box representing coil and α-helix, respectively.

FIG. 2: Purification of CBL TKBD and SLAP2. A) Absorbance chromatograms (280 nm) for CBL TKBD (solid line) and mSLAP2 (hatched line) proteins subjected to gel filtration analysis individually and mixed in a 1:1 molar ratio (dotted line). B) SDS-PAGE gel stained with Coomassie blue of fractions 10-22 from gel filtration analysis of the CBL/SLAP2 complex.

FIG. 3: Crystal structure of CBL and SLAP2. A) Electron density at the site of CBL and SLAP2 interaction is shown in grey, with CBL and SLAP2 atoms shown in stick. Carbon atoms are coloured according to their respective backbones, with CBL monomers in blue and SLAP2 monomers in green. Oxygen and nitrogen atoms are coloured red and blue, respectively. For clarity, portions of CBL in the plane of the page have been removed. B) Ribbon representations of the Cα atoms of the CBL/SLAP2 crystal structure, coloured as in A, with elements of CBL TKBD labelled. Panels are related by a rotation of approximately 90° about the horizontal axis. C) Superposition of the Cα atoms of the 4H bundle and EF-hand of CBL/SLAP2 with those of unliganded (PDB id: 1B47) and liganded CBL TKBD (PDB id: 2Cbl), shown in blue, cyan, and magenta, respectively. All ribbon diagrams were prepared with Pymol (The PyMOL Molecular Graphics System, Version 1.5.0.4 Schrödinger, LLC.).

FIG. 4: CBL dimerization and unassigned electron density. A) Ribbon representation of the Cα atoms of the CBL TKBD crystal structure, with molecules 1 and 2 shown in light and dark blue, respectively. B) Ribbon representation of the Cα atoms of a representative CBL structure (PDB id:3BUW), with molecules 1 and 2 shown in cyan and orange, respectively, and pTyr peptides shown in green and magenta. Molecule 1 is in approximately the same orientation as that in A. C) Electron density at the CBL dimer interface is shown in grey, with the Cα trace of CBL molecules 1 and 2 shown as light and dark blue lines. D) As in C, rotated approximately 90° about the horizontal axis. E) Cα traces of CBL TKBD crystal structure, coloured as in C, with symmetry related molecules as calculated by Coot shown in orange. F) As in E, different orientation.

FIG. 5: Validation of CBL/SLAP2 interaction by mutagenesis. A) Ribbon representation of the Cα atoms of the CBL/SLAP2 crystal structure, with a pTyr peptide modeled in magenta. Subdomains of CBL TKBD are shown, with the 4H bundle in teal, EF-hand in dark blue, and SH2 domain in light blue. SLAP2 is coloured green. B) As in A, showing the CBL regulatory cleft. C) Magnification of the CBL/SLAP2 interface, coloured as in A, with side chains of CBL, SLAP2 molecule 1, and SLAP2 molecule 2 shown in stick and labelled in teal/dark blue/light blue, black, and green, according to their respective backbones. Oxygen, nitrogen, and sulfur atoms are coloured red, blue, and yellow, respectively. D) SDS-PAGE gel stained with Coomassie blue for CBL and SLAP2 proteins co-purified under high salt (upper panel) and low salt (lower panel) conditions from Duet-His-Δlinker-hSLAP2-CBL and its mutated variants. SLAP2 and CBL mutants are labelled in black and blue, respectively. E) As in D, for SLAP2/SLAP2 interface mutants, co-purified in low salt conditions. F) As in D, for CBL and SLAP proteins co-purified in low salt conditions from Duet-His-mSLAP-CBL and its mutated variants.

FIG. 6: CBL/SLAP2 binding interface. Ribbon representation of the Cα atoms of the CBL/SLAP2 crystal structure, with the 4H bundle, EF-hand, and SH2 domain coloured teal, dark blue, and light blue, respectively. SLAP2 is coloured green. Side chains shown in stick are coloured according to their respective backbones. Oxygen, nitrogen, and sulfur atoms are coloured red, blue, and yellow, respectively.

FIG. 7: Phosphorylation of SLAP2 at tyrosine 248. A) SDS-PAGE gel stained with Coomassie blue for SLAP2 and SLAP2 incubated with EphA4 kinase under phosphorylating conditions (left panel). Immunoblot of SLAP2 +/- EphA4, immunoblotted with anti-pTyr (right panel). B) As in A, with EphA4 alone also shown. Box indicates upper gel band excised for mass spectrometry analysis. C) Intensity versus charge to mass ratio (m/z) plot for SLAP2 C-tail peptides ESLSSY₂₄₈ISLAEDPLDDA (upper panel) and SLSSY₂₄₈ISLAEDPLDDA (lower panel) generated by protease digestion of gel band from B with trypsin and GluC, respectively, followed by LC-MS/MS analysis. D) SDS-PAGE gel stained with Coomassie blue for CBL and SLAP2 WT or Y248F co-purified under low salt conditions from Duet-His-Δlinker-hSLAP2-CBL expressed in normal (BL21) or phosphorylating (TKB1) conditions. E) Ribbon representation of the Cα atoms of the CBL/SLAP2 interface, with CBL shown in blue and SLAP2 in green. Residues of CBL and SLAP2 are shown in stick with carbon atoms coloured according to their respective backbones, and oxygen, nitrogen, sulfur, and phosphorus atoms coloured red, blue, yellow, and orange, respectively. A phosphate group has been modeled on the side-chain hydroxyl group of tyrosine 248.

FIG. 8: SLAP2 binding precludes LHR-RING interactions with the TKBD. A) Superposition of the Cα atoms of CBL/SLAP2 with CBL TKBD-LHR-RING (PDB id:2Y1N) and B) TKBD-LHR-RING plus E2 (PDB id:1FBV). CBL/SLAP2 is shown in dark blue and green, superpositioned on CBL TKBD-LHR-RING shown in beige, yellow, and orange, respectively. E2, pTyr peptide, and calcium ions are shown in cyan, magenta, and mauve, respectively. C) Magnification of the CBL regulatory cleft, with Leu241 of SLAP2 (green) and Tyr271 of CBL LHR (yellow) shown in stick (PDB id:2Y1N).

FIG. 9: SLAP2 stimulates CBL ubiquitination activity in vitro. A) In vitro ubiquitination reactions containing CBL TKBD-LHR-RING (CBL) or phosphorylated CBL (pCBL), incubated for 100 minutes with or without SLAP2 or phosphorylated SLAP2 (pSLAP2), analyzed by SDS-PAGE and immunoblotted with anti-ubiquitin antibody. The right panel shows the Fast Green stained PVDF membrane of the transferred reactions. B) As in A, with reactions stopped over a time course of 80 minutes. C) Histogram showing fold change in ubiquitination activity compared to unphosphorylated CBL using E3LITE assay. The pCBL reaction was diluted 2.7 fold to give a comparable detection range on the plate reader. A representative experiment performed in triplicate is shown with error bars representing standard deviation (SD). Biological replicates with independent protein preparations yielded consistent changes in ubiquitination activity and are shown in FIG. 10A. D) In vitro ubiquitination reactions were analyzed by LC-MS/MS. Stacked histogram of ubiquitin linkages K11, K48, and K63, shown as a percentage of the total number of linkages detected in each reaction.

FIG. 10 : Ubiquitination activity in vitro. A) Histogram showing fold change in ubiquitination activity of pCBL or CBL plus SLAP2 or pSLAP2 compared to unphosphorylated CBL using E3LITE assay. The pCBL reaction was diluted 2.7 fold to give a comparable detection range on the plate reader. A representative experiment performed in triplicate is shown with error bars representing SD. B) As in A), for SLAP2 WT and mutants. C) In vitro ubiquitination reactions containing CBL or pCBL, incubated with or without SLAP2 or pSLAP2, WT or mutants, analyzed by SDS-PAGE and immunoblotted with anti-Ub antibody. D-F) As in A), for CBL and SLAP2 WT and mutants.

FIG. 11: Mutation of CBL/SLAP2 interface reduces CBL ubiquitination activity. A) In vitro ubiquitination reactions containing CBL or pCBL, incubated with or without SLAP2 or pSLAP2, WT or mutants (L241R, S244E), analyzed by SDS-PAGE and immunoblotted with anti-Ub antibody. The right panel shows the Fast Green stained PVDF membrane of the transferred reactions. B) Histogram showing fold change in ubiquitination activity in reactions containing CBL and SLAP2 or pSLAP2, WT or mutants, compared to unphosphorylated CBL using E3LITE assay. A representative experiment performed in triplicate is shown with error bars representing SD. Biological replicates with independent protein preparations yielded consistent changes in ubiquitination activity and are shown in FIG. 10B. C) and D) As in A and B, respectively, comparing CBL WT and G306E incubated with SLAP2 or pSLAP2. Biological replicates with independent protein preparation yielded consistent changes in ubiquitination activity and are shown in FIG. 10E.

FIG. 12: CBL substrate ubiquitination is regulated by SLAP2 binding . A) In vitro ubiquitination reactions containing CBL or pCBL WT or mutants (A223R, S226E, and double mutant AR/SE), analyzed by SDS-PAGE and immunoblotted with anti-Ub antibody. The right panel shows the Fast Green stained PVDF membrane of the transferred reactions. B) Histogram showing fold change in ubiquitination activity of CBL mutants compared to unphosphorylated CBL using E3LITE assay. Error bars represent SD. Biological replicates with independent protein preparations yielded consistent changes in ubiquitination activity and are shown in FIG. 10F. C) Immobilized GST-CBL or GST-CBL with TKBD mutations A223R, S226E or AR/SE, were incubated with EGFR transfected COS-7 cell protein lysates. Bound proteins were resolved by SDS-PAGE and immunoblotted with anti-EGFR. D) Full length HA-CBL WT or AR/SE mutant was co-transfected with FLAG-EGFR and His-Ub in HEK 293T cells. Equivalent protein lysates were resolved by SDS-PAGE and immunoblotted with anti-His to detect ubiquitinated species. E) Full length HA-CBL WT or AR/SE mutant was co-transfected with FLAG-EGFR and His-Ub in HEK 293T cells. EGFR was immunoprecipitated with anti-FLAG, resolved by SDS-PAGE and immunoblotted with anti-His.

FIG. 13: Screen work flow and details.

FIG. 14: Hit Determination. CBL activating hits were determined by luminescent signal at least 4 standard deviations from the mean of all compounds on a 384 well plate. An example with 2 hits identified is shown.

### DETAILED DESCRIPTION OF CERTAIN ASPECTS

### Definitions

Unless otherwise explained, all technical and scientific terms used herein have the same meaning as commonly understood by one of ordinary skill in the art to which this disclosure belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Although any methods and materials similar or equivalent to those described herein can be used in the practice for testing of the present invention, the typical materials and methods are described herein. In describing and claiming the present invention, the following terminology will be used.

It is also to be understood that the terminology used herein is for the purpose of describing particular aspects only, and is not intended to be limiting. Many patent applications, patents, and publications are referred to herein to assist in understanding the aspects described. Each of these references are incorporated herein by reference in their entirety.

In understanding the scope of the present application, the articles “a”, “an”, “the”, and “said” are intended to mean that there are one or more of the elements. Additionally, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

It will be understood that any aspects described as “comprising” certain components may also “consist of” or “consist essentially of,” wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Typically, a composition consisting essentially of a set of components will comprise less than 5% by weight, typically less than 3% by weight, more typically less than 1%, and even more typically less than 0.1% by weight of non-specified component(s).

It will be understood that any component defined herein as being included may be explicitly excluded from the claimed invention by way of proviso or negative limitation.

In addition, all ranges given herein include the end of the ranges and also any intermediate range points, whether explicitly stated or not.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The abbreviation, “e.g.” is derived from the Latin exempli gratia, and is used herein to indicate a non-limiting example. Thus, the abbreviation “e.g.” is synonymous with the term “for example.”

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “expression” as used herein is defined as the transcription and/or translation of a particular nucleotide sequence driven by its promoter.

“lsolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, typically, a human.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Parenteral” administration of a composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein’s or peptide’s sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The terms “therapeutically effective amount”, “effective amount” or “sufficient amount” mean a quantity sufficient, when administered to a subject, including a mammal, for example a human, to achieve a desired result, for example an amount effective to cause a protective immune response. Effective amounts of the compounds described herein may vary according to factors such as the immunogen, age, sex, and weight of the subject. Dosage or treatment regimes may be adjusted to provide the optimum therapeutic response, as is understood by a skilled person. For example, administration of a therapeutically effective amount of the compositions described herein is, in aspects, sufficient to inhibit CBL autoinhibition. In other aspects, administration of a therapeutically effective amount of the compositions described herein is sufficient to treat a disease or condition, such as cancer, for example, leukemia.

Moreover, a treatment regime of a subject with a therapeutically effective amount may consist of a single administration, or alternatively comprise a series of applications. The length of the treatment period depends on a variety of factors, such as the agent for use in the treatment, the age of the subject, the concentration of the agent, the responsiveness of the patient to the agent, or a combination thereof. It will also be appreciated that the effective dosage of the agent used for the treatment may increase or decrease over the course of a particular treatment regime. Changes in dosage may result and become apparent by standard diagnostic assays known in the art. The compositions described herein may, in aspects, be administered before, during or after treatment with conventional therapies for the disease or disorder in question, such as cancer. For example, the compositions described herein may find particular use in combination with chemotherapy.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

The term “subject” as used herein refers to any member of the animal kingdom, typically a mammal. The term “mammal” refers to any animal classified as a mammal, including humans, other higher primates, domestic and farm animals, and zoo, sports, or pet animals, such as dogs, cats, cattle, horses, sheep, pigs, goats, rabbits, etc. Typically, the mammal is human.

Administration “in combination with” one or more further therapeutic agents includes simultaneous (concurrent) and consecutive administration in any order.

The term “pharmaceutically acceptable” means that the compound or combination of compounds is compatible with the remaining ingredients of a formulation for pharmaceutical use, and that it is generally safe for administering to humans according to established governmental standards, including those promulgated by the United States Food and Drug Administration.

The term “pharmaceutically acceptable carrier” includes, but is not limited to solvents, dispersion media, coatings, antibacterial agents, antifungal agents, isotonic and/or absorption delaying agents and the like. The use of pharmaceutically acceptable carriers is well known.

“Variants” are biologically active peptides or fragments thereof having an amino acid sequence that differs from a comparator sequence by virtue of an insertion, deletion, modification and/or substitution of one or more amino acid residues within the comparative sequence. Variants generally have less than 100% sequence identity with the comparative sequence. Ordinarily, however, a biologically active variant will have an amino acid sequence with at least about 70% amino acid sequence identity with the comparative sequence, such as at least about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity. The variants include peptide fragments of at least 3 amino acids that retain some level of the biological activity of the comparator sequence. Variants also include polypeptides wherein one or more amino acid residues are added at the N- or C-terminus of, or within, the comparative sequence. Variants also include polypeptides where a number of amino acid residues are deleted and optionally substituted by one or more amino acid residues. Variants also may be covalently modified, for example by substitution with a moiety other than a naturally occurring amino acid or by modifying an amino acid residue to produce a non-naturally occurring amino acid.

“Percent amino acid sequence identity” is defined herein as the percentage of amino acid residues in the candidate sequence that are identical with the residues in the sequence of interest, such as the polypeptides of the invention, after aligning the sequences and introducing gaps, if necessary, to achieve the maximum percent sequence identity, and not considering any conservative substitutions as part of the sequence identity. None of N-terminal, C-terminal, or internal extensions, deletions or insertions into the candidate sequence shall be construed as affecting sequence identity or homology. Methods and computer programs for the alignment are well known in the art, such as “BLAST”.

A substantially identical sequence may comprise one or more conservative amino acid mutations. It is known in the art that one or more conservative amino acid mutations to a reference sequence may yield a mutant peptide with no substantial change in physiological, chemical, or functional properties compared to the reference sequence; in such a case, the reference and mutant sequences would be considered “substantially identical” polypeptides. Conservative amino acid mutation may include addition, deletion, or substitution of an amino acid; a conservative amino acid substitution is defined herein as the substitution of an amino acid residue for another amino acid residue with similar chemical properties (e.g. size, charge, or polarity).

In a non-limiting example, a conservative mutation may be an amino acid substitution. Such a conservative amino acid substitution may substitute a basic, neutral, hydrophobic, or acidic amino acid for another of the same group. By the term “basic amino acid” it is meant hydrophilic amino acids having a side chain pK value of greater than 7, which are typically positively charged at physiological pH. Basic amino acids include histidine (His or H), arginine (Arg or R), and lysine (Lys or K). By the term “neutral amino acid” (also “polar amino acid”), it is meant hydrophilic amino acids having a side chain that is uncharged at physiological pH, but which has at least one bond in which the pair of electrons shared in common by two atoms is held more closely by one of the atoms. Polar amino acids include serine (Ser or S), threonine (Thr or T), cysteine (Cys or C), tyrosine (Tyr or Y), asparagine (Asn or N), and glutamine (Gln or Q). The term “hydrophobic amino acid” (also “non-polar amino acid”) is meant to include amino acids exhibiting a hydrophobicity of greater than zero according to the normalized consensus hydrophobicity scale of Eisenberg (1984). Hydrophobic amino acids include proline (Pro or P), isoleucine (lle or l), phenylalanine (Phe or F), valine (Val or V), leucine (Leu or L), tryptophan (Trp or W), methionine (Met or M), alanine (Ala or A), and glycine (Gly or G). “Acidic amino acid” refers to hydrophilic amino acids having a side chain pK value of less than 7, which are typically negatively charged at physiological pH. Acidic amino acids include glutamate (Glu or E), and aspartate (Asp or D).

Sequence identity is used to evaluate the similarity of two sequences; it is determined by calculating the percent of residues that are the same when the two sequences are aligned for maximum correspondence between residue positions. Any known method may be used to calculate sequence identity; for example, computer software is available to calculate sequence identity. Without wishing to be limiting, sequence identity can be calculated by software such as NCBI BLAST2 service maintained by the Swiss Institute of Bioinformatics (and as found at ca.expasy.org/tools/blast/), BLAST-P, Blast-N, or FASTA-N, or any other appropriate software that is known in the art.

“Active” or “activity” for the purposes herein refers to a biological activity of the agents described herein, wherein “biological” activity refers to a biological function (either inhibitory or stimulatory) caused by the agents.

### Agents

Described herein are agents that inhibit CBL autoinhibition. In other words, the agents relieve the autoinhibitory state of CBL. This is typically accomplished by mimicking the action of SLAP/SLAP2 or by enhancing the activity of native SLAP/SLAP2. As described, SLAP/SLAP2 has been identified as a regulator of CBL activity and interaction between SLAP/SLAP2 and CBL results in activation of CBL through inhibiting its own autoinhibition mechanisms.

The agents typically interact with CBL in one or more CBL domains, such as a tyrosine-kinase binding domain (TKBD), a linker helix region (LHR), or a RING domain of CBL. The agents described herein typically act independently of phosphorylation, meaning that they do not phosphorylate CBL as part of their mechanism of action in inhibiting CBL autoinhibition. They may have a secondary phosphorylation activity in some aspects, while in other aspects, they do not have any phosphorylation activity. For example, in some aspects, the agents described herein do not bind to the phospho-tyrosine binding site of the TKBD, even if they do interact with the TKBD in other ways, such as through interacting with a region of CBL that is distinct from the phospho-tyrosine binding site of the TKBD.

Typically, the agents described herein interact with the SLAP and/or SLAP2 binding cleft, also referred to herein as the CBL regulatory cleft. The CBL regulatory cleft is typically framed by one or more of the 4H bundle, the EF-hand, and the SH2 domain of CBL. More specifically, the CBL regulatory cleft is typically framed by helices αC and αD of the 4H bundle, helix αE2 and loop αE2-αF2 of the EF-hand, and helix αN, loop αN-βA, and strand βA of the SH2 domain.

As noted above, the agents described herein may be SLAP and/or SLAP2 mimetics. Exemplary methods of designing and testing mimetics for SLAP/SLAP2-driven CBL inhibitory activity are described herein and will be understood by a skilled person. For example, a high-throughput screen for novel compounds that mimic SLAP and/or SLAP2 binding is described and identified 10 exemplary SLAP and/or SLAP2 mimetics. In this particular screen, the agents identified were peptidomimetics and include N-[(2-chloro-6-fluorophenyl)methyl]-8-methyl-3,4-dihydro-2H-1,5-benzodioxepine-7-carboxamide; N-(3-bromophenyl)-5-(5-cyclobutyl-1,3,4-oxadiazol-2-yl)thiophene-2-sulfonamide; 3-chloro-N-{3-cyclobutyl-[1,2,4]triazolo[4,3-a]pyridin-8-yl}-4-methoxybenzene-1-sulfonamide; N-[(4-methoxyphenyl)methyl]-3-methyl-1-[3-(trifluoromethyl)benzenesulfonyl]piperidine-3-carboxamide; N-[4-chloro-3-(trifluoromethyl)phenyl]-4-(5-cyclobutyl-1,2,4-oxadiazol-3-yl)thiophene-2-sulfonamide; 5-(2-cyclobutyl-1,3-oxazol-5-yl)-N-[4-(trifluoromethoxy)phenyl]thiophene-2-sulfonamide; 1-(4-methylbenzenesulfonyl)-N-(naphthalen-1-yl)-5-oxopyrrolidine-2-carboxamide; 2-{4-[(4-methylphenyl)methyl]-2,3-dioxopiperazin-1-yl}-N-[4-(propan-2-yl)phenyl]acetamide; N-(2-{4-[4-(5-cyclobutyl-1,2,4-oxadiazol-3-yl)phenyl]piperazin-1-yl}-2-oxoethyl)furan-2-carboxamide; and 5-oxo-3-phenyl-N-[3-(propan-2-yloxy)propyl]-5H-[1,3]thiazolo[3,2-a]pyrimidine-6-carboxamide; and combinations thereof.

It will be understood that the agents are not limited to peptidomimetics and that the agents described herein may be of any form known to a skilled person. For example, the agent may comprise or consist of a peptide, a polynucleotide, a small molecule, a lipid, a carbohydrate, or a combination of any of these moieties. Combinations of such moieties will be understood. As an example, peptides can be linked to small molecules or can be glycosylated. Other combinations are contemplated herein either as linked molecules or as separate molecules to be used simultaneously in separate compositions or in a single composition, or sequentially one after the other.

For example, if the agent is a peptide, the peptide may be used as part of an antibody or fragment thereof, the peptide may be a linear, cyclic, or branched peptide or a combination thereof, the peptide may be a glycopeptide, the peptide may be used as one part of a fusion peptide, the peptide may be a stapled peptide, or the peptide may be a peptidomimetic. Combinations of these classes of peptides are contemplated herein.

In other aspects, if the agent is a polynucleotide, it will be understood that the polynucleotide may comprise DNA, RNA, or combinations thereof. The polynucleotide may be used for gene therapy, for example, and may be administered alone or packaged in a viral construct or in any other known manner for delivery of DNA and/or RNA. The polynucleotide may be an antisense or sense nucleotide and it may target SLAP and/or SLAP2 directly or indirectly through other mediators in order to enhance SLAP and/or SLAP2 activity. Further, SLAP and/or SLAP2 constructs may be used to enhance the number of SLAP/SLAP2 molecules available within a subject for interacting with CBL and decreasing CBL autoinhibition.

In other aspects, if the agent is a small molecule, any type or class of small molecule is contemplated. In a specific aspect, the small molecule is a macrocyclic compound.

Any of the agents described herein may be further linked to one or more small molecules or other agents, such as a drug, imaging, or targeting agent. For example, a peptide may be used to relieve CBL autoinhibition and a small molecule linked to the peptide may be used as a secondary agent to treat the condition associated with CBL autoinhibition. In other aspects, the agent, such as a peptide or small molecule, may be linked to an antibody that helps target the agent to a desired organ or a site of a tumor, for example. In other aspects, the agent may be linked to an imaging agent so that the agent can be tracked using imaging after administration.

The agents described herein may be incorporated into a delivery system, such as a gene therapy platform, a liposome, a nanoparticle, a therapeutic cell treatment, or a combination thereof. A skilled person will understand how to incorporate various agents into different delivery systems, as desired. Such delivery systems may aid in increasing bioavailability, in targeting the agent to the desired site of treatment, or in increasing the half-life of the agent, for example.

In specific aspects, the agents described herein may be SLAP and/or SLAP2 mimetics. As noted above, the mimetic may be a peptide, a polynucleotide, including DNA and/or RNA, a small molecule, such as a macrocyclic compound, a lipid, a carbohydrate, or a combination thereof. Exemplary peptides include antibodies or fragments thereof, linear, cyclic, or branched peptides or combinations thereof, glycopeptides, a fusion peptides, stapled peptides, peptidomimetics, or combinations thereof. The mimetic may further be linked to a small molecule, such as a drug, imaging, or targeting agent.

In other specific aspects, the agents described herein are recombinant SLAP and/or SLAP2 or a variant and/or fragment thereof. The variant or fragment thereof is active and inhibits CBL autoinhibition. Typically, the variant or fragment thereof comprises at least about 70%, 75%, 80%, 85%, 90%, 95%, 96%, 97%, 98%, 99%, or 99.5% sequence identity to the native SLAP and/or SLAP2 sequence:

MGNSMKSTPAPAERPLPNPEGLDSDFLAVLSDYPSPDISPPIFRRGEKLR
VISDEGGWWKAISLSTGRESYIPGICVARVYHGWLFEGLGRDKAEELLQL
PDTKVGSFMIRESETKKGFYSLSVRHRQVKHYRIFRLPNNWYYISPRLTF
QCLEDLVNHYSEVADGLCCVLTTPCLTQSTAAPAVRASSSPVTLRQKTVD
WRRVSRLQEDPEGTENPLGVDESLFSYGLRESIASYLSLTSEDNTSFDRK
KKSISLMYGGSKRKSSFFSSPPYFED (SLAP; SEQ IDNO:1)

and/or

MGSLPSRRKSLPSPSLSSSVQGQGPVTMEAERSKATAVALGSFPAGGPAE
LSLRLGEPLTIVSEDGDWWTVLSEVSGREYNIPSVHVAKVSHGWLYEGLS
REKAEELLLLPGNPGGAFLIRESQTRRGSYSLSVRLSRPASWDRIRHYRI
HCLDNGWLYISPRLTFPSLQALVDHYSELADDICCLLKEPCVLQRAGPLP
GKDIPLPVTVQRTPLNWKELDSSLLFSEAATGEESLLSEGLRESLSFYIS
LNDEAVSLDDA (SLAP2; SEQ ID NO:2).

The fragment may be of any length that retains some level of activity with respect to inhibiting CBL autoinhibition. Typically, the fragment comprises from about 3 to about 275 amino acids for SLAP and from about 3 to about 260 amino acids for SLAP2, and any range thereinbetween. For example, the fragment in aspects comprises from about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 180, about 185, about 190, about 195, about 200, about 205, about 210, about 215, about 220, about 225, about 230, about 235, about 240, about 245, about 250, about 255, about 260, about 265, or about 270 amino acids to about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 180, about 185, about 190, about 195, about 200, about 205, about 210, about 215, about 220, about 225, about 230, about 235, about 240, about 245, about 250, about 255, about 260, about 265, about 270, or about 275 amino acids.

In other aspects, the fragment comprises at least about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 180, about 185, about 190, about 195, about 200, about 205, about 210, about 215, about 220, about 225, about 230, about 235, about 240, about 245, about 250, about 255, about 260, about 265, or about 270 amino acids.

When the agent is recombinant SLAP and/or SLAP2 or a variant or fragment thereof, as above, the recombinant molecule may be in the form of an antibody or fragment thereof, a linear, cyclic, or branched peptide or a combination thereof, a glycopeptide, a fusion peptide, a stapled peptide, a peptidomimetic, or a combination thereof. Similarly, the recombinant molecule may be linked to a small molecule, such as a drug, imaging, or targeting agent.

In other specific aspects, the agent described herein is a fusion protein, wherein one moiety of the fusion protein comprises a peptide agent described herein, such as a mimetic or recombinant SLAP/SLAP2 molecule. The fusion protein may comprise a second such peptide agent as described herein or some other molecule such as a therapeutic peptide, an imaging peptide, a targeting peptide, or a combination thereof.

The agents described herein may include modifications. Such modifications include, but are not limited to, conjugation to an effector molecule such as a chemotherapeutic agent or a targeting moiety. Modifications further include, but are not limited to conjugation to detectable reporter moieties. Modifications that extend half-life (e.g., pegylation) are also included. Proteins and non-protein agents may be conjugated to the agents by methods that are known in the art. Conjugation methods include direct linkage, linkage via covalently attached linkers, and specific binding pair members (e.g., avidin-biotin). Such methods include, for example, that described by Greenfield et al., Cancer Research 50, 6600-6607 (1990), which is incorporated by reference herein and those described by Amon et al., Adv. Exp. Med. Biol. 303, 79-90 (1991) and by Kiseleva et al, Mol. Biol. (USSR)25, 508-514 (1991), both of which are incorporated by reference herein.

The polypeptides of the present invention may also comprise additional sequences to aid in their expression, detection or purification. Any such sequences or tags known to those of skill in the art may be used. For example, and without wishing to be limiting, polypeptides may comprise a targeting or signal sequence (for example, but not limited to ompA), a detection tag, exemplary tag cassettes include Strep tag, or any variant thereof; see, e.g., U.S. Pat. No. 7,981,632, His tag, Flag tag having the sequence motif DYKDDDDK, Xpress tag, Avi tag,Calmodulin tag, Polyglutamate tag, HA tag, Myc tag, Nus tag, Stag, SBP tag, Softag 1, Softag 3, V5 tag, CREB-binding protein (CBP), glutathione S-transferase (GST), maltose binding protein (MBP), green fluorescent protein (GFP), Thioredoxin tag, or any combination thereof; a purification tag (for example, but not limited to a His₅ or His₆), or a combination thereof.

In another example, the additional sequence may be a biotin recognition site such as that described by Cronan et al in WO 95/04069 or Voges et al in WO/2004/076670. As is also known to those of skill in the art, linker sequences may be used in conjunction with the additional sequences or tags.

More specifically, a tag cassette may comprise an extracellular component that can specifically bind to an antibody with high affinity or avidity. Within a single chain fusion protein structure, a tag cassette may be located (a) immediately amino-terminal to a connector region, (b) interposed between and connecting linker modules, (c) immediately carboxy-terminal to a binding domain, (d) interposed between and connecting a binding domain (e.g., scFv or scFab) to an effector domain, (e) interposed between and connecting subunits of a binding domain, or (f) at the amino-terminus of a single chain fusion protein. In certain embodiments, one or more junction amino acids may be disposed between and connecting a tag cassette with a hydrophobic portion, or disposed between and connecting a tag cassette with a connector region, or disposed between and connecting a tag cassette with a linker module, or disposed between and connecting a tag cassette with a binding domain.

Also encompassed herein are isolated or purified polypeptides or fragments thereof immobilized onto a surface using various methodologies; for example, and without wishing to be limiting, the polypeptides may be linked or coupled to the surface via His-tag coupling, biotin binding, covalent binding, adsorption, and the like. The solid surface may be any suitable surface, for example, but not limited to the well surface of a microtiter plate, channels of surface plasmon resonance (SPR) sensorchips, membranes, beads (such as magnetic-based or sepharose-based beads or other chromatography resin), glass, a film, or any other useful surface.

In other aspects, the agents described herein may be linked to a cargo molecule; the agents may deliver the cargo molecule to a desired site and may be linked to the cargo molecule using any method known in the art (recombinant technology, chemical conjugation, chelation, etc.). The cargo molecule may be any type of molecule, such as a therapeutic or diagnostic agent. For example, and without wishing to be limiting in any manner, the therapeutic agent may be a radioisotope, which may be used for radioimmunotherapy; a toxin, such as an immunotoxin; a cytokine, such as an immunocytokine; a cytotoxin; an apoptosis inducer; an enzyme; an anti-cancer antibody for immunotherapy; or any other suitable therapeutic molecule known in the art. In the alternative, a diagnostic agent may include, but is by no means limited to a radioisotope, a paramagnetic label such as gadolinium or iron oxide, a fluorophore, a Near InfraRed (NIR) fluorochrome or dye (such as Cy3, Cy5.5, Alexa680, Dylight680, or Dylight800), an affinity label (for example biotin, avidin, etc), fused to a detectable protein-based molecule, or any other suitable agent that may be detected by imaging methods. In a specific, non-limiting example, agent may be linked to a fluorescent agent such as FITC or may genetically be fused to the Enhanced Green Fluorescent Protein (EGFP).

Also described herein are polynucleotides encoding the polypeptides described herein, as well as vectors comprising the polynucleotides and host cells comprising the vectors. For example, expression vectors are provided containing the polynucleotide sequences operably linked to an expression sequence, a promoter and an enhancer sequence. A variety of expression vectors for the efficient synthesis of polypeptides in prokaryotic, such as bacteria, and eukaryotic systems, including but not limited to yeast and mammalian cell culture systems have been developed. The vectors can comprise segments of chromosomal, non-chromosomal and synthetic DNA sequences.

The polypeptides described herein can be fused to additional amino acid residues. Such amino acid residues can be a peptide tag to facilitate isolation, for example. Other amino acid residues for homing of the antibodies to specific organs or tissues are also contemplated.

### Methods of Screening

Also described herein are methods of screening for agents that inhibit CBL autoinhibition and/or agents that are SLAP and/or SLAP2 mimetics. The method typically comprises applying the agent to be tested to a composition comprising CBL followed by detecting a change in CBL activation based on measuring assembly of polyubiquitin chains by CBL. Compounds that mimic SLAP and/or SLAP2 binding inhibit the autoinhibited state of CBL, favoring the activated state leading to E2 binding and assembly of polyubiquitin chains, which is associated with E3 ligase activity. Thus, an increase in CBL activation suggests that the agent inhibits CBL autoinhibition and/or is a SLAP and/or SLAP2 mimetic. Typically, the CBL comprises the TKBD-LHR-RING region and the CBL is typically recombinant CBL.

While agents can be tested individually or in small groups, typically, the method is a high-throughput method.

The identified hits are typically validated by retesting hits and assembling a titration curve using the same assay. An independent assay using an anti-ubiquitin immunoblot rather than the plate based assay can be used to validate the agents identified as hits.

Also described herein are agents identified by the method of screening.

### Methods of Use

In another aspect, described herein are methods of treating subjects by administering a therapeutically effective amount of the agents described herein to a subject in need thereof, as well as uses of the agents described herein for treating a subject. Therapeutically effective means an amount effective to produce the desired therapeutic effect, such as treating cancer.

For example, described herein are methods of inhibiting CBL autoinhibition. These methods comprise administering an agent as described herein to a subject in need thereof. Typically, the methods are for treating a disease or condition in which CBL inhibition or downregulation is implicated. It will be appreciated that this could be any disease or condition resulting from CBL and/or SLAP or SLAP2 mutations or other effects that result in aberrant CBL autoinhibition or reduction in CBL activation, or conditions in which tyrosine kinase activity is increased, whether congenital or associated with cancer for example.

Examples of diseases or conditions in which CBL inhibition or downregulation is implicated include cancer, and other diseases including moyamoya angiopathy and Noonan syndrome. Cancers in which CBL is implicated include, for example, leukemia, such as AML, JMML, CMML, or CML, lung cancer, or head and neck cancer. Cancers in which tyrosine kinase activity is implicated may also be targeted by the methods described herein.

Any suitable method or route can be used to administer agents described herein. Routes of administration include, for example, oral, intravenous, intraperitoneal, subcutaneous, or intramuscular administration.

The agents described herein, where used in a subject for the purpose of prophylaxis or treatment, are typically administered in the form of a composition additionally comprising a pharmaceutically acceptable carrier. Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the binding proteins. The compositions of the injection may, as is well known in the art, be formulated so as to provide quick, sustained or delayed release of the active ingredient after administration to the subject.

The above disclosure generally describes the present invention. A more complete understanding can be obtained by reference to the following specific examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The following examples may not include detailed descriptions of conventional methods, such as those employed in the construction of vectors and plasmids, the insertion of genes encoding polypeptides into such vectors and plasmids, or the introduction of plasmids into host cells. Such methods are well known to those of ordinary skill in the art and are described in numerous publications including Sambrook, J., Fritsch, E. F. and Maniatis, T. (1989), Molecular Cloning: A Laboratory Manual, 2nd edition, Cold Spring Harbor Laboratory Press, which is incorporated by reference herein.

Without further description, it is believed that one of ordinary skill in the art can, using the preceding description and the following illustrative examples, make and utilize the claimed agents, mimetics, and peptides and practice the claimed methods and uses. The following working examples, therefore, specifically point out the typical aspects and are not to be construed as limiting in any way the remainder of the disclosure.

### EXAMPLES

### Example 1: Structural Basis for Phosphorylation Independent Activation of C-CBL by SRC-Like Adaptor Protein 2

### Abstract

CBL is a RING type E3 ubiquitin ligase that functions as a central regulator of tyrosine kinase signaling and loss of CBL function is implicated in several forms of leukemia. The Src-like adaptor proteins (SLAP/SLAP2) bind to CBL and are key components of CBL-dependent downregulation of antigen receptor, cytokine receptor, and receptor tyrosine kinase signaling in hematopoietic cells. To understand the molecular basis of the interaction between SLAP/SLAP2 and CBL, we solved the crystal structure of CBL TKBD in complex with SLAP2. The carboxy-terminal region of SLAP2 adopted an α-helical structure which bound in a cleft between the 4H, EF-hand, and SH2 domains of CBL, opposite the phospho-tyrosine peptide binding site. Tyrosine residue 248 in the SLAP2 C-terminal tail was identified as a site of phosphorylation, and addition of phosphorylated SLAP2 to autoinhibited CBL was found to activate CBL ubiquitination activity. Disruption of the SLAP2/CBL interface through mutagenesis revealed a novel regulation mechanism for CBL autoubiquitination activity. Our results indicate that SLAP2 adaptor binding to CBL is an additional mechanism for regulation of CBL ubiquitination activity.

### Introduction

Ubiquitin (Ub) modification of proteins regulates cellular processes including cell cycle, DNA repair, endocytosis, and signal transduction. The ubiquitination machinery consists of a cascade of E1, E2, and E3 components, which activate and transfer ubiquitin to substrates in a sequential manner. Substrate specificity in the ubiquitin system is determined through a large and diverse family of E3 ubiquitin ligases that bind target proteins. The Casitas B-cell lymphoma (CBL) gene encodes the E3 ubiquitin ligase CBL (also referred to as c-CBL), a central regulator of tyrosine kinase (TK) signaling. Ubiquitination of activated receptor TKs by CBL nucleates the assembly of endocytic proteins both at the membrane and at sorting endosomes to promote lysosome targeting, degradation and signal termination. CBL is also important for down regulation of signaling from antigen and cytokine receptors through ubiquitination of receptor chains and associated cytosolic TKs, leading to inactivation and/or proteosomal degradation.

CBL consists of an amino terminal tyrosine kinase-binding domain (TKBD), a linker helix region (LHR) and a really interesting new gene (RING) domain, followed by a carboxy-terminal region containing binding sites for Src homology 2 (SH2) and Src homology 3 (SH3) domain containing signaling and adaptor proteins. CBL TKBD is composed of a four-helix bundle (4H), an EF hand, and a variant SH2 domain, which binds substrates, such as activated TKs, in a phospho-tyrosine dependent manner. The CBL RING domain binds E2 conjugating enzymes required for ubiquitin transfer to TKBD bound substrates. Recruitment of CBL to activated TK complexes leads to phosphorylation at a conserved tyrosine residue (Tyr371) within the CBL LHR, and activation of its E3 ligase function. Structural studies have shown that unphosphorylated CBL adopts a closed autoinhibited conformation with Tyr371 forming part of a LHR-TKBD interface. Phosphorylation of Tyr371 releases the LHR-TKBD interaction causing a conformational change that places the E2 active site in close proximity to TKBD bound substrate, and stabilizing E2 bound ubiquitin for transfer.

Mutations in CBL which impair E3 ligase activity have been identified in juvenile myelomonocytic leukemia (JMML), chronic myelomonocytic leukemia (CMML), and chronic myeloid leukemia (CML), as well as in patients with several other myeloproliferative neoplasms. Understanding CBL autoinhibition and phosphorylation-dependent activation provide insight into how mutations identified in leukemias can abrogate CBL E3 ligase activity. In addition to mutations in the RING domain, the LHR, and Tyr371 in particular, are frequent targets of missense mutations that prevent phosphorylation dependent activation of CBL ubiquitin ligase function. These mutant forms of CBL are also thought to act through a gain-of-function mechanism, since adaptor functions are retained, allowing signaling protein recruitment through multiple carboxy-terminal SH2 and SH3 binding motifs.

Src-like adaptor proteins (SLAP and SLAP2) bind CBL and play important roles in CBL activity and regulation of antigen receptor, cytokine receptor, and RTK signaling. Both Cbl and Slap deficient mice have increased T and B cell receptor (TCR and BCR) levels, accompanied by enhanced signaling and positive selection of thymocytes. Slap/Slap2 deficient mice also show defective down regulation of Granulocyte/macrophage colony-stimulating factor receptor (GM-CSFR) signaling leading to a block in normal dendritic cell development, as well as enhanced platelet activation signaling and arterial thrombus formation. In addition, SLAP/SLAP2 overexpression or depletion in hematopoietic cell lines affects the down regulation of several RTKs, including CSF-1R, c-Kit and FLT3. Outside the hematopoietic system, SLAP is important for down regulation of SRC and EPHA2 signaling in intestinal epithelial cells.

SLAP and SLAP2 are membrane associated proteins by virtue of an amino-terminal myristoylation site. They contain adjacent SH3 and SH2 domains most closely related to those found in SRC family kinase HCK, followed by a carboxyl (C)-terminal tail region lacking obvious domains or protein interaction motifs. The SLAP2 SH3/SH2 domains adopt typical folds with a uniquely short connector sequence that positions them in close association consistent with a functional unit that binds proteins through tandem association. SLAP/SLAP2 recruitment to activated receptor TKs is mediated through the SH3/SH2 module, while the C-terminal tail region of both SLAP and SLAP2 constitutively associates with CBL. SLAP association with CBL is important for ubiquitin dependent down regulation of ZAP-70 and TCRζ, as well as RTKs FLT3 and c-KIT.

While CBL binds to multiple SH2 and SH3 domain containing adaptor proteins through canonical binding motifs found in the CBL carboxy-terminal region, SLAP and SLAP2 binding requires a region of the SLAP/SLAP2 C-terminal tail and the CBL TKBD. Nevertheless, SLAP/SLAP2 binding to CBL is distinct from other TKBD binding proteins as it is independent of tyrosine phosphorylation and is not disrupted by a CBL Gly306Glu mutation, which abolishes TKBD binding to tyrosine-phosphorylated substrates. To understand the molecular basis of this interaction, we solved the X-ray crystal structure of CBL TKBD in complex with SLAP2. We describe how a C-terminal region of SLAP2 adopts an α-helical structure that binds CBL TKBD in the same cleft that is occupied by the LHR in the autoinhibited CBL conformation. Furthermore, we find that SLAP2 binding can stimulate CBL autoubiquitination activity independent of phosphorylation indicating that in addition to its adaptor functions, SLAP/SLAP2 binding contributes to CBL activation.

### Results

### Crystal Structure of CBL TKBD in Complex With SLAP2

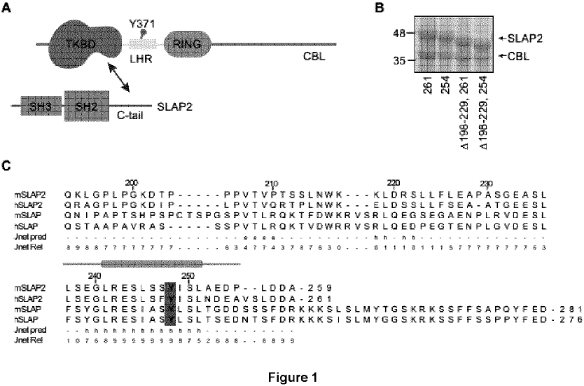

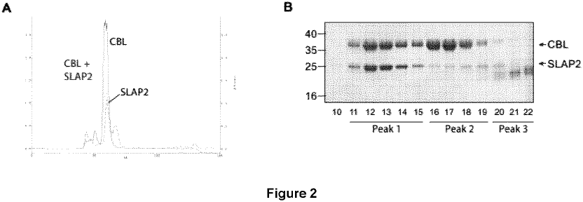

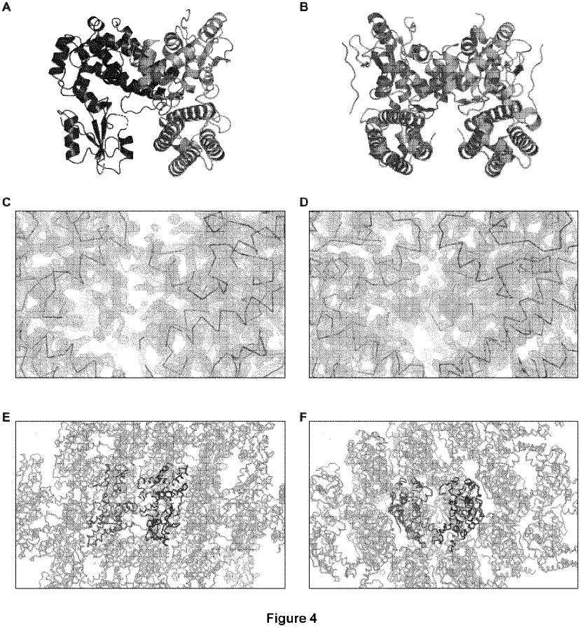

SLAP2 interacts with the CBL TKBD via a phosphorylation independent mechanism involving its C-terminal region (FIG. 1A). To understand the molecular basis of this interaction, CBL TKBD and SLAP2 proteins (residues 25-357 and 28-261, respectively) were purified by affinity and size exclusion chromatography. To form a complex of CBL TKBD and SLAP2 for co-crystallization studies, the purified proteins were mixed in a 1:1 molar ratio and subjected to size exclusion chromatography. The proteins eluted as two peaks, one at an earlier volume than typically observed for either CBL TKBD or SLAP2, indicating higher molecular weight (FIG. 2A). As confirmed by SDS-PAGE of chromatography fractions, the first peak contained a CBL/SLAP2 complex (FIG. 2B), which was concentrated and used for sparse matrix crystallization experiments. Small rod-like crystals diffracted to 2.5 Å resolution, but structure determination by molecular replacement using native CBL TKBD (PDB ID: 2Y1M) and SLAP2 SH3-SH2 (PDB ID: 4M4Z) as models proved unsuccessful, as no solution was found for SLAP2. Using CBL TKBD as a model resulted in a traceable electron density map and refineable model of CBL TKBD. Comparison of the CBL TKBD structure to published or released CBL models in the protein data bank (PDB) revealed no significant changes in overall conformation or secondary structure. However, it was noted that our model of CBL represented a novel mode of configuration or packing of CBL molecules in the crystal asymmetric unit (ASU) (FIGS. 4A, B). Close examination of the interface between the two CBL molecules in the ASU revealed unassigned electron density that clearly resembled two alpha helices (FIGS. 4C, D). We reasoned that this density likely represented a portion of SLAP2.

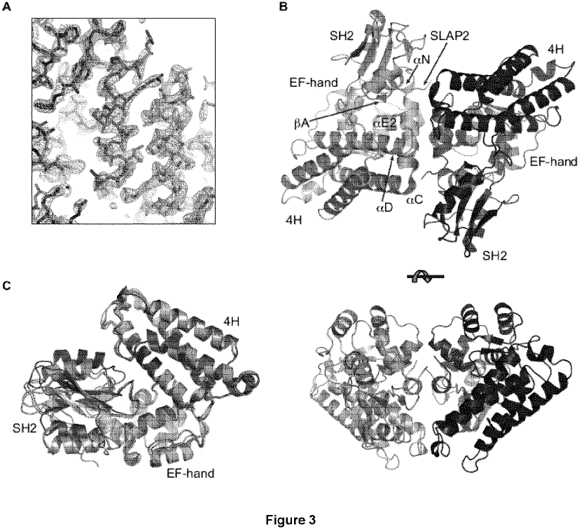

To identify which residues to assign to the helical electron density, iterative rounds of model refinement and data processing were employed, resulting in improved electron density maps with recognizable side chain densities. Based on observation that the C-terminal tail of SLAP2, excluding residues 198-229 and 255-261, is necessary for CBL binding in vitro (FIG. 1B), the fact that large portions of the SLAP2 C-terminal tail are expected to be disordered by secondary structure prediction programs (FIG. 1C), and the contour of the electron density, residues 237-255 of SLAP2 were manually placed into each helix of the unassigned density (FIG. 3A). The resulting model refined readily with good quality refinement statistics (Table 1) signifying that a structure of CBL TKBD in complex with SLAP2 had been determined (FIG. 3B). It was not possible to establish if additional SLAP2 residues co-crystallized with CBL TKBD, but remained disordered. However, based on protein packing within the crystal (FIGS. 4E, F) and our observation that SLAP2 is susceptible to degradation, we conjecture that CBL/SLAP2 co-crystallized following degradation of the C-terminal tail from the SH3-SH2 domain, and that residues 237-255 represent the critical component for CBL/SLAP2 interaction.

TABLE 1

Data collection and refinement statistics
Data collection

| Space group | P2₁ |
|---|---|
| Cell dimensions | |
| a, b, c (Å) | 62.7, 87.0, 65.3 |
| α, β, γ (°) | 90, 112.3, 90 |
| Resolution (Å) | 45.3-2.5 |
| R_{merge} | 8.0 (60.1)* |
| \|/σ\| | 8.3 (1.5) |
| Completeness (%) | 90.5 (57.8) |
| Redundancy | 2.9 (2.0) |
| Refinement | |
| Resolution (Å) | 45.3–2.5 |
| No. reflections 18867 | 18867 |
| Rwork/Rfree | 24.9/29.4 |
| Number of non-hydrogen atoms | |
| Macromolecules | 4954 |
| Ligands | 2 |
| Solvent | 47 |
| Average B-factors (Å²) | 48.5 |
| R.m.s deviations | |
| Bond lengths (Å) | 0.002 |
| Bond angles (°) | 0.602 |

Molprobty Statistics

| | |
|---|---|
| All-atom clashscore | 6.0 |
| Ramachandran | |
| Favored (%) | 93.5 |
| Allowed (%) | 99.2 |

*Highest resolution shell is shown in parenthesis.

Superposition of CBL/SLAP2 with native CBL TKBD (PDB id: 1B47) indicates that CBL adopts the typical integrated module, composed of a calcium-bound EF-hand wedged between a four-helix bundle (4H) and a divergent SH2 domain, with root mean square deviation (rmsd) of 0.9 Å (main chain atoms) for CBL TKBD (FIG. 3C). Superposition of the 4H bundle and EF-hand of CBL/SLAP2 with those of native and liganded CBL TKBD (PDB id: 2CBL) shows that binding of the C-terminal portion of SLAP2 to CBL does not induce closure of the domains to the extent that phosphopeptide binding to the SH2 domain does, such that CBL/SLAP2 most resembles unliganded CBL TKBD (FIG. 3C).

### Structure of CBL/SLAP2 Defines a Novel Binding Interface

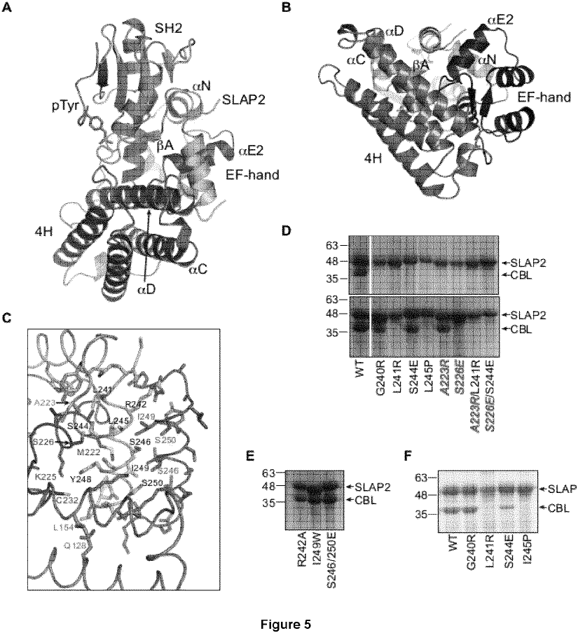

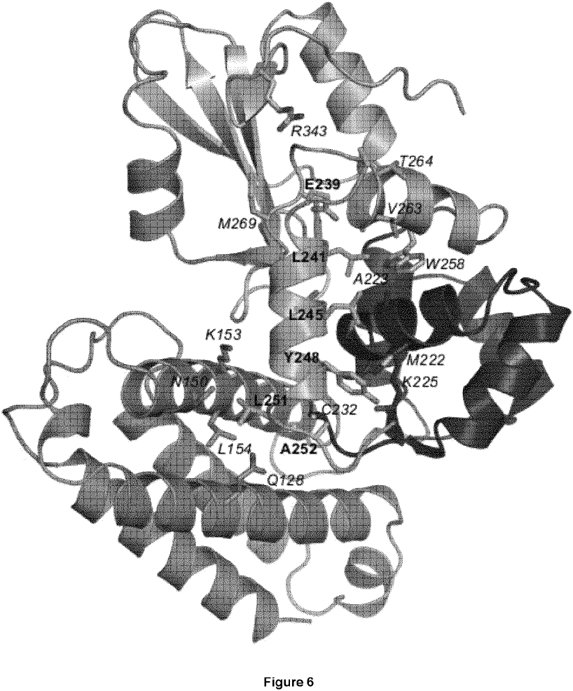

SLAP2 binds as an α-helix in a cleft formed by the three subdomains of TKBD, opposite the phospho-tyrosine peptide binding pocket (FIG. 5A). The SLAP2 binding cleft is framed by helices αC and αD of the 4H bundle, helix αE2 and loop αE2-αF2 of the EF-hand, and helix αN, loop αN-βA, and strand βA of the SH2 domain (FIG. 5A). The CBL/SLAP2 interface is stabilized by hydrophobic interactions involving side chains from Leu241, Leu245, and Leu251 of SLAP2 and Lys153, Leu154, Met222, Ala223, Trp258, and Val263 of CBL, and by hydrogen bonds involving both backbone carbonyl groups and side chain atoms (see Table 2 and FIG. 6). Altogether, the interaction between CBL and SLAP2 results in 4592 Å² of overall contact area. The crystal ASU contains two CBL TKBD molecules and two SLAP2 molecules related by a two-fold symmetry axis to form a CBL/LAP2 dimer (FIG. 3B). The CBL TKBD structure is well ordered except for the N-terminal (25-47 molecule 1, 25-51 molecule 2) and C-terminal (353-357 molecule 1, 352-357 molecule 2) residues. The SLAP2 helices interact expansively, stabilized by extensive hydrogen bonding and hydrophobic interactions, while few interactions are formed between the CBL monomers (Tables 3 and 4).

TABLE 2

Interactions between SLAP2 and CBL

| SLAP2 Molecule 1 | CBL Molecule 1 | CBL Secondary Structure Element | Distance (Å) |
|---|---|---|---|
| Leu 237 O | Val 263 O | αN-βA loop | 2.8 |
| Leu 237 CD1 | Arg 343 NH2 | SH2 C-terminus | 3.4 |
| Leu 237 CB | Thr 264 CA | αN-βA loop | 4.6 |
| Glu 239 OE1 | Ala 262 O | helix αN | 3.0 |
| Glu 239 OE1 | Val 263 O | αN-βA loop | 2.8 |
| Glu 239 OE2 | Ala 262 O | helix αN | 2.8 |
| Gly 240 CA | Met 269 CA | strand βA | 4.4 |
| Gly 240 O | Ala 270 CB | strand βA | 3.8 |
| Leu 241 CD1 | Trp 258 CZ2 | helix αN | 4.0 |
| Leu 241 CD1 | Val 263 CG2 | αN-βA loop | 3.6 |
| Leu 241 CD2 | Ala 223 CB | helix αE2 | 3.7 |
| Leu 241 N | Tyr 268 CE2 | strand βA | 4.1 |
| Ser 244 OG | Ser 226 OG | helix αE2 | 3.8 |
| Leu 245 CG | Ala 223 CA | helix αE2 | 4.3 |
| Leu 245 CD2 | Met 222 CE | helix αE2 | 3.7 |
| Tyr 248 OH | Lys 225 NZ | helix αE2 | 3.2 |
| Tyr 248 CD1 | Cys 232 SG | αE2-αF2 loop | 4.8 |
| Leu 251 CD1 | Lys 153 CB | helix αD | 3.7 |
| Leu 251 CD2 | Gln 128 NE2 | helix αC | 3.8 |
| Leu 251 CD2 | Asn 150 OD1 | helix αD | 3.6 |
| Leu 251 CD2 | Leu 154 CD2 | helix αD | 4.7 |
| Leu 251 O | Gln 128 NE2 | helix αC | 3.2 |
| Ala 252 CB | Cys 232 SG | αE2-αF2 loop | 4.2 |

TABLE 3

Interactions between SLAP2 molecules

| SLAP2 Molecule 1 | SLAP2 Molecule 2 | Distance (Å) |
|---|---|---|
| Arg 242 NE | lle 249 O | 3.3 |
| Arg 242 NH2 | Ala 252 O | 2.5 |
| Arg 242 NH1 | Ser 250 O | 2.9 |
| Arg 242 NE | Asp 254 OD | 3.1 |
| Leu 245 CB | lle 249 CG2 | 4.1 |
| Leu 245 CB | lle 249 CD1 | 3.7 |
| Leu 245 CD1 | lle 249 CD1 | 3.9 |
| Ser 246 OG | Ser 250 OG | 3.2 |
| lle 249 CG2 | Leu 245 CB | 3.9 |
| lle 249 CD1 | Leu 245 CB | 3.7 |
| lle 249 CD1 | Leu 245 CD1 | 3.8 |
| lle 249 O | Arg 242 NH1 | 2.6 |
| Ser 250 OG | Ser 246 OG | 4.2 |
| Ala 252 O | Arg 242 NH2 | 3.2 |
| Asp 254 OD2 | Arg 242 NH1 | 2.6 |

TABLE 4

Interactions between CBL molecules

| CBL Molecule 1 | CBL Molecule 2 | Distance (Å) |
|---|---|---|
| Glu 135 CA | Phe 284 CZ | 8.1 |
| Glu 135 CA | Lys 283 CA | 10.2 |
| Lys 137 CA | Lys 283 CA | 11.3 |
| Glu 143 O | Glu 143 O | 3.8 |
| Asn 144 CB* | Ser 145 CA | 4.5 |
| Asn 144 ND2* | Gln 146 N | 3.6 |
| Ser 145 CA | Asn 144 CB | 5.3 |
| Gln 146 N | Asn 144 ND2* | 4.3 |
| Leu 219 CA | Leu 219 CD2 | 3.8 |
| Leu 219 CD2 | Leu 219 CA | 3.8 |
| Leu 219 CD2 | Met 222 SD | 3.6 |
| Met 222 SD | Leu 219 CD2 | 4.4 |
| Met 222 SD | Met 222 SD | 5.9 |
| Lys 283 CA | Lys 137 CA | 11.1 |
| Lys 283 CB | Glu 135 CA | 7.4 |
| Phe 284 CZ | Glu 135 CA | 9.0 |

*Electron density absent for these atoms

To validate the CBL/SLAP2 binding interface, a series of mutants in a Duet co-expression vector system was generated for co-purification trials. The SLAP2 sequence was preceded by thioredoxin (Trx) and His-tag fusion proteins to allow co-purification of CBL TKBD that was bound to Trx-His-SLAP2 during the initial affinity chromatography purification step. Surface residues deemed to be important to the CBL/SLAP2 interface, but not CBL function or folding, were mutated to disrupt CBL/SLAP2 binding (FIG. 5C and FIG. 6). In our structure, SLAP2 Gly240 locates close to CBL Met269 and Ala270, such that mutation to a bulky Arg residue (G240R) could cause sterical constraints. Similarly, SLAP2 Leu241 is situated in a hydrophobic cleft composed of CBL Ala223, Trp258, and Val263; mutation to Arg (L241R) was predicted to disturb this binding pocket. Mutation of CBL Ala223 to Arg (A223R) would likewise disrupt this pocket, with CBL/SLAP2 double mutant A223R/L241R predicted to drastically disturb binding through sterical constraints and charge repulsion. (For clarity, CBL mutations herein are indicated in italics, SLAP2 in regular font.) Similarly, single or double mutation of CBL Ser226 or SLAP2 Ser244, whose side chain hydroxyl groups are in close proximity to one another, to charged Glu residues (S226E and S244E) was anticipated to interfere with CBL/SLAP2 binding. Finally, given that SLAP2 binds CBL as an α-helix, mutation of Leu245, located in the center of this α-helix, to proline (L245P) was expected to interrupt the helical structure and thus prevent SLAP2 binding. CBL TKBD co-purified with SLAP2 in high salt conditions (0.5 M NaCl), while mutation of either SLAP2 or CBL at interface residues resulted in loss of CBL co-purification (FIG. 5D, upper panel). Co-purification in less stringent low salt concentration (0.15 M NaCl) revealed that mutation at L241R, L245P, and S226E still abrogated CBL co-purification, while G240R, S244E, and A223R mutations were tolerated, resulting in CBL co-purification (FIG. 5D, lower panel). However, double mutation of the CBL/SLAP2 interface (A223R/L241R and S226E/S244E; FIG. 5D, lower panel) was not tolerated, even at low salt concentration. In contrast, mutating SLAP2 residues involved in the SLAP2/SLAP2 interface but not CBL binding (R242A, I249W, S246/250E) did not significantly impact co-purification of CBL, suggesting that TKBD binding does not require a SLAP2 dimer (FIG. 5E).

The related adaptor protein SLAP also interacts with CBL TKBD, and its amino acid sequence corresponding to the SLAP2 C-terminal α-helical region is highly conserved (FIG. 1C). To determine if SLAP binds CBL TKBD by a similar mechanism, we tested SLAP and a set of analogous interface mutants in co-purification experiments. Like SLAP2, SLAP mutations at L241R and I245P abrogated CBL co-purification, while mutations G240R and S224E were tolerated (FIG. 5F). Together, these studies indicate that SLAP and SLAP2 interact with CBL via an α-helix near their C-terminus, which binds to a cleft of the CBL TKBD that is distinct from the canonical phospho-tyrosine binding site in the CBL SH2 domain.

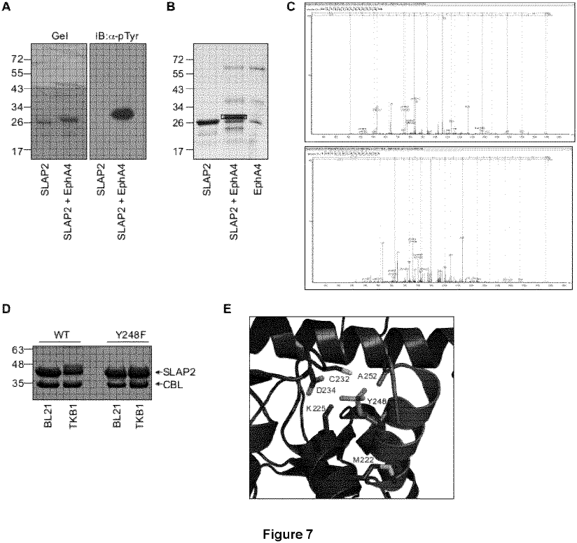

Further examination of the SLAP/SLAP2 α-helix protein sequence revealed a conserved tyrosine residue (Tyr248) (FIG. 1C) that is predicted to be phosphorylated by the Kinexus phosphorylation site prediction algorithm, but to our knowledge has not been experimentally confirmed (www.phosphonet.ca). SLAP2 has been shown to be tyrosine phosphorylated in CSF-1 stimulated FD-Fms cells. To identify SLAP2 tyrosine phosphorylation sites, purified SLAP2 protein was incubated in vitro with EphA4 kinase in the presence of ATP. Phosphorylated SLAP2 was detected by an upward shift in molecular weight by SDS-PAGE and confirmed by immunoblot analysis (FIG. 7A). Sites of phosphorylation were identified by in-gel protease digestion of the isolated, shifted band followed by liquid chromatography tandem mass spectrometry (LC-MS/MS), which identified Tyr248 as a site of phosphorylation (FIGS. 7B,C). To investigate whether phosphorylation of Y248 impacts SLAP2 binding to CBL, co-purification experiments were performed under conditions where SLAP2 was tyrosine phosphorylated. CBL TKBD co-purified with phosphorylated SLAP2 WT and Y248F indicating that SLAP2 interaction with CBL is not disrupted by Y248 phosphorylation, nor is it required for binding (FIG. 7D). In agreement, a phosphate ion was modeled on the side chain hydroxyl of Tyr248 in the CBL/SLAP2 structure (FIG. 7E). The structure appears to accommodate phosphorylation at Tyr248 without causing sterical constraints at the CBL/SLAP2 interface.

### SLAP2 Binding to CBL Precludes Inhibitory LHR-RING Interactions With the TKBD

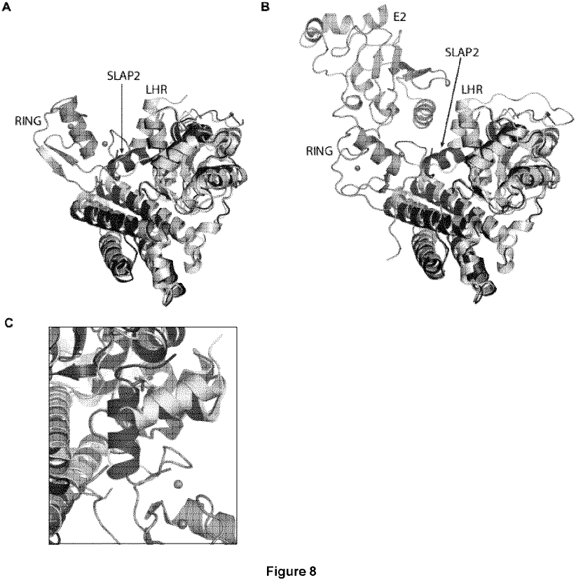

Unphosphorylated CBL adopts a closed, autoinhibited conformation in which the LHR and RING domain pack against the TKBD in a manner that restricts ubiquitin ligase activity. Phosphorylation of Tyr371 in the LHR disrupts these interactions, reorients the LHR-RING in proximity of substrates, and stabilizes E2 bound ubiquitin for transfer. Superposition of CBL/SLAP2 with structures of autoinhibited CBL (TKBD-LHR-RING, PDB id:2Y1N and TKBD-LHR-RING plus E2, PDB id:1 FBV) revealed that SLAP2 binds to a similar region of CBL TKBD as the regulatory LHR and RING domain (FIGS. 8A,B). The LHR forms an ordered loop and an α-helix that pack against the TKBD, with linker-TKDB interactions centered on linker residues Tyr368 and Tyr371. In the CBL/SLAP2 structure, SLAP2 residue Leu241 occupies the same buried environment as Tyr371 in autoinhibited CBL (FIG. 8C). Moreover, side chain residues from helix αE2 of the EF-hand, such as Ala223 and Ser226, as well as Val263 from loop αN-βA of the SH2 domain, make multiple van der Waals contacts with the linker helix in autoinhibited CBL; these same residues are involved in the SLAP2 binding site (FIG. 5C, FIG. 6, Table 2). Similarly, the SLAP2 α-helix and its C-terminal extension occupy the same space as linker-loop 2 of the LHR and the N-terminus of the RING domain in closed CBL, respectively (FIG. 8C). Indeed, several residues which stabilize the TKBD-RING interface in closed CBL, including Gln128, Asn150, Lys153, and Leu154, are involved in SLAP2 binding in the CBL/SLAP2 structure (FIG. 6, Table 2). CBL residue Met222 stabilizes the TKBD-RING interface in autoinhibited CBL and forms part of an E2 binding pocket upon the addition of UbcH7. Notably, Met222 is also involved in hydrophic interactions with Leu245 and lle249 of the SLAP2 α-helix. These observations suggest that SLAP2 binding to CBL would preclude LHR-RING interactions with the TKBD and favour the open, catalytically competent conformation.

### Binding of SLAP2 C-Tail to CBL Regulatory Cleft Promotes CBL Ubiquitin Ligase Activity

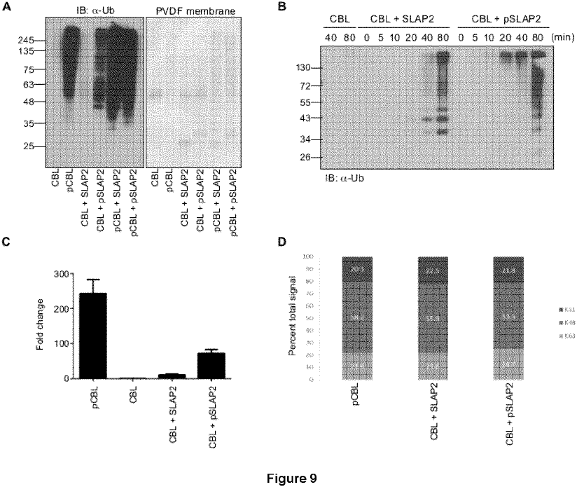

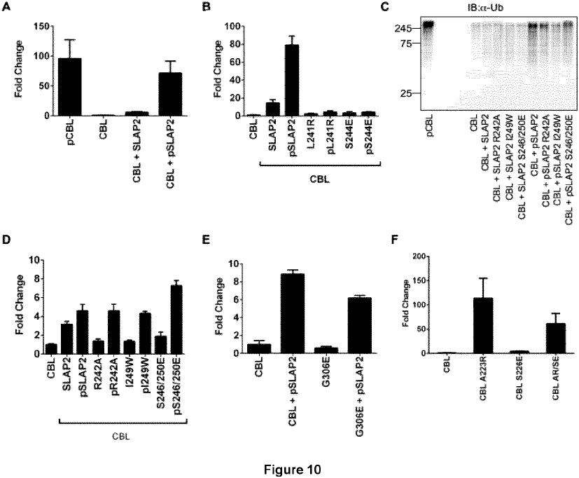

Given that binding of SLAP2 and the LHR-RING would be mutually exclusive (FIG. 8C), we reasoned that binding of SLAP2 to CBL could displace the LHR-RING, thereby promoting the catalytically competent conformation. To test this hypothesis, an in vitro ubiquitination assay in which E3 ligase activity is detected by a smear at high molecular weight on an anti-ubiquitin (Ub) immunoblot was employed. As previously reported, purified CBL TKBD-LHR-RING (2-436) had no detectable ubiquitination activity, while its phosphorylated version (pCBL) displayed substantial activity, indicating the inactive and active forms of CBL, respectively (FIG. 9A). Addition of purified SLAP2 or phosphorylated SLAP2 (pSLAP2) to unphosphorylated CBL stimulated CBL ubiquitination activity in vitro (FIG. 9A). Notably, addition of pSLAP2 to CBL appeared to promote CBL ubiquitination activity to a greater extent than the addition of SLAP2 (FIGS. 9A,B). Neither addition of SLAP2 nor pSLAP2 to active pCBL appeared to further enhance or diminish ubiquitination activity (FIG. 9A). To further compare and quantify CBL ubiquitin ligase activity, a chemiluminescence based assay which measures the total amount of polyubiquitylated products formed in an in vitro reaction was used. In this assay, production of polyubiquitylated proteins increased upon addition of SLAP2 or pSLAP2 to CBL, compared to CBL alone (FIG. 9C and FIG. 10A). Consistent with the anti-Ub immunoblot analysis, pSLAP2 stimulated CBL ligase activity to a greater extent than SLAP2. Notably, the ubiquitin ligase activity of CBL/pSLAP2 remained far less than that of pCBL (FIGS. 9A,C). This suggests that pSLAP2 interaction with CBL is partially activating, such that a catalytically competent conformation is favoured upon SLAP2 binding.

Ubiquitin contains seven internal lysine residues (K6, K11, K27, K29, K33, K48 and K63) that can be modified with additional Ub molecules to form extended oligomers or “chains” (Kerscher, Felberbaum, Hochstrasser Annu Rev Cell Dev Biol 2006; Hong, Ng, Srikumar, Raught Proteomics 2015). To further examine the impact of pSLAP2 on CBL ubiquitination activity, we determined the type of Ub linkages generated in vitro by pCBL or CBL in the presence of SLAP2 or pSLAP2, as different types of Ub chains can confer different biological outcomes. Reactions from the in vitro ubiquitination assay were analyzed by LC-MS/MS. Mass spectrometry data was searched against the human proteome, with diglycine as a variable modification on lysine, and the MS1 peak area corresponding to each linkage type quantified (Hong et al, 2015). Phosphorylated CBL generated predominantly K48 linked Ub chains with smaller proportions of K11 and K63 linkages (FIG. 9D). Similar proportions of Ub chain linkages were observed when unphosphorylated CBL was incubated with either SLAP2 or pSLAP2 (FIG. 9D) indicating that the in vitro ubiquitination activity of CBL in the presence of SLAP2 or pSLAP2 is similar to that of phosphorylated CBL.

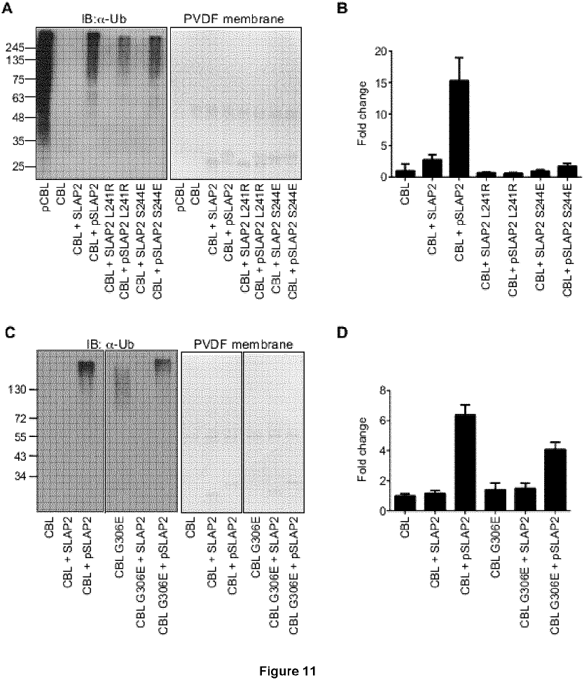

To assess the specific role of SLAP2 α-helix in CBL activation, we tested the ability of SLAP2 mutants L241R and S244E to promote CBL ligase activity. Phosphorylated SLAP2 L241R exhibited reduced stimulation of CBL ubiquitination activity compared to pSLAP2 WT (FIGS. 11A,B and FIG. 10B). Phosphorylated SLAP2 S244E, which maintained CBL binding under low salt conditions (FIG. 5D) such as those used in the ubiquitination assay, also exhibited less stimulation of CBL ubiquitination activity than pSLAP2 WT (FIGS. 11A,B and FIG. 10B). In contrast, SLAP2 mutants R242A, I249W, and S246/250E, which do not interfere with CBL/SLAP2 binding but are predicted to disrupt the dimer interface of SLAP2 α-helices observed in our structure, did not alter SLAP2 or pSLAP2 activation of CBL (FIGS. 10C,D). This suggests that activation of CBL by SLAP2 requires binding of the SLAP2 C-terminal tail α-helix to the CBL TKBD surface identified in the CBL/SLAP2 crystal structure. Given this CBL interface is also involved in maintaining CBL in an inactive state, through interactions with its LHR and RING domain, we refer to this region as the CBL regulatory cleft.

Engagement of tyrosine phosphorylated substrates by CBL TKBD leads to reorientation of the RING domain and consequently increased E2 binding. Thus, we tested whether the ability of SLAP2 to promote CBL ubiquitination activity is influenced by canonical TKBD-phospho-tyrosine interactions. CBL mutant G306E, which abolishes phospho-tyrosine binding in vitro, behaved similarly to WT CBL in ubiquitination assays, such that pSLAP2 also stimulated CBL G306E ubiquitination activity (FIGS. 11C,D and FIG. 10E). This indicates that SLAP2 activaton of CBL is independent of both CBL Tyr371 phosphorylation and an intact CBL TKBD substrate binding domain.

### Phosphorylation Independent Activation of CBL Promotes Substrate Ubiquitination

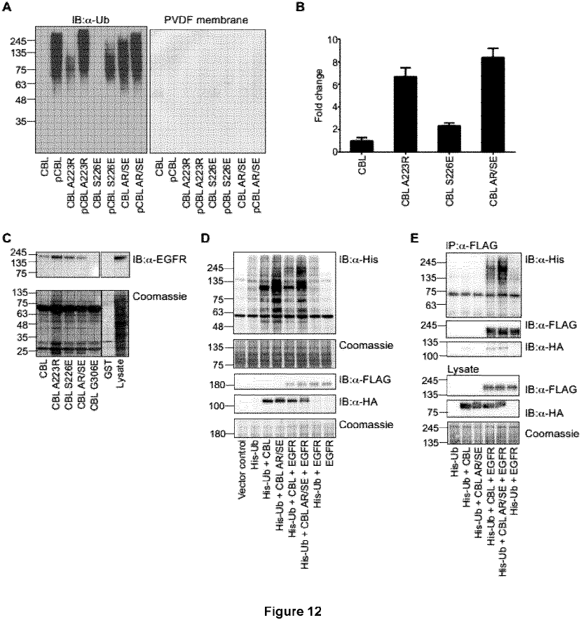

Next, the ubiquitination activity of CBL proteins mutated in the CBL regulatory cleft was compared to CBL WT. Strikingly, mutations A223R, S226E, and A223R/S226E (AR/SE) lead to activation of unphosphorylated CBL, compared to WT, suggesting that mutations in the SLAP2 binding cleft also disrupt the interaction between CBL TKBD and the LHR (FIGS. 12A,B and FIG. 10F). These data indicate that mutations of CBL which disrupt SLAP2 binding may also disrupt the autoinhibitory LHR-TKBD interaction and confer phosphorylation independent activation.

To investigate the role of CBL/SLAP2 interaction in cells, the effect of the CBL A223R/S226E mutation on substrate binding and ubiquitination was examined. First, to confirm that the AR/SE mutation does not disrupt TKBD folding and binding to phospho-tyrosine substrates, glutathione-S-transferase (GST)-tagged WT or mutant CBL TKBD-LHR-RING fusion proteins were isolated on beads and incubated with lysates from COS-7 cells stimulated with epidermal growth factor (EGF). Captured proteins bound to either CBL WT or regulatory cleft mutants were analyzed by anti-EGF receptor (EGFR) immunoblot (FIG. 12C). CBL regulatory cleft mutants A223R, S226E and AR/SE all retained the ability to bind activated EGFR, indicative of functional TKBD phospho-tyrosine binding, whereas the SH2 mutant G306E did not bind EGFR. Next, the A223R/S226E mutation was introduced into full length HA-tagged CBL and co-transfected into HEK293 cells with His-Ub and FLAG-tagged EGFR. Protein lysates from cells expressing CBL AR/SE showed an increased incorporation of His-Ub compared to cells expressing CBL WT (FIG. 8D) with or without EGFR co-expression. Furthermore, ubiquitination of immunoprecipitated EGFR was enhanced when co-expressed with CBL AR/SE compared to CBL WT (FIG. 12E). Together these data support a role for SLAP2 binding and the TKBD regulatory cleft in promoting CBL ubiquitination of TK substrates.

### Discussion

Compared to other phospho-tyrosine binding domains such as PTB and SH2 domains, the more complex architecture of CBL TKBD is indicative of additional protein interaction sites and regulatory regions. Indeed, the CBL/SLAP2 structure reveals the presence of an additional intermolecular protein interaction surface formed by regions of the SH2 domain, EF hand and four-helix bundle that is distinct from the phospho-tyrosine binding site. Other adaptor proteins shown to bind CBL TKBD, such as APS and Sprouty, are phospho-tyrosine and SH2 domain dependent, while SLAP2 binding involves a unique surface of TKBD and is independent of phosphorylation.

Three mammalian CBL proteins are encoded by separate genes: CBL (also known as c-Cbl and studied here), Cbl-b, and Cbl-c. CBL and CBL-b proteins are highly homologous in the TKBD and Cbl-b is also regulated by phosphorylation of LHR tyrosine residue (Y363), which relieves autoinhibition to enhance ubiquitin ligase activity. The residues involved in SLAP2 binding as identified by our CBL/SLAP2 crystal structure are conserved in Cbl-b TKBD and SLAP has been shown to bind Cbl-b in vitro. Therefore, SLAP/SLAP2 binding may also provide a mechanism for phosphorylation independent activation of Cbl-b.

CBL E3 ligase function is integral to regulation of tyrosine kinase signaling through ubiquitination of tyrosine phosphorylated substrates. Furthermore, CBL activity is controlled by tyrosine phosphorylation which regulates the equilibrium between the autoinhibited and catalytically open conformational states. Our study reveals that SLAP/SLAP2 adaptor binding provides an additional mechanism to regulate CBL E3 ligase activity. The structure of SLAP2 bound to CBL TKBD showed that the SLAP2 binding site overlaps the region bound by the LHR in the autoinhibited conformation. Therefore, SLAP2 adaptor binding could hinder access to the closed conformation, shifting the equilibrium toward the activated state. However, SLAP2 binding does not stimulate CBL ubiquitination activity to the same magnitude as phosphorylation of CBL at Tyr371. This suggests that the effect of SLAP2 binding on the equilibrium between closed and open conformations is dynamic, presumably due to the absence of phosphorylation of LHR residue Tyr371, which serves to stabilize the active conformation of CBL. While the aromatic ring of pTyr371 forms hydrophobic interactions with other LHR residues in active CBL, the phosphate moiety forms crucial hydrophilic interactions with Lys382 and Lys389 of the RING domain. This suggests that SLAP2 interaction with CBL is partially activating, such that an open catalytically competent conformation is favoured, but lacks the pTyr371 interactions with the RING domain that stabilize E2 - ubiquitin binding and optimize k_{cat} for CBL autoubiquitination.

Protein binding as a means to regulate activity has previoulsy been observed for E3 ligases, including CBL family proteins. Cbl-c ubiquitination activity is increased by interaction of its RING finger domain with a LIM domain of Hic-5, a member of the paxillin family. However, in contrast to our observations for activation of CBL by SLAP2 binding, Hic-5 can increase the activity of Cbl-c only after the E3 is activated by phosphorylation or phosphomimetic mutation at LHR tyrosine Y341. The HECT E3 Smurf2 also adopts an autoinhibited structure that is relieved by binding an adaptor protein, Smad7. Smad7 regulates the ubiquitin ligase activity of Smurf2 by promoting E2 binding to the HECT domain. Thus, adaptor protein binding as a means to regulate E3 activity may be a more general mechanism.

Our data explains the requirement the SLAP adaptor protein in CBL dependent down-regulation of TK signaling in specific cellular contexts. Analysis of SLAP deficient mice have highlighted its involvement in T and B cell development, dendritic cell maturation, platelet activation and mast cell degranulation. The phenotypes observed were shown to be a consequence of defective down regulation of tyrosine kinase linked receptors including TCR, BCR, GM-CSFR. In addition, studies in cell lines demonstrated that the SLAP/SLAP2 C-terminal domain is required for CBL dependent down regulation of TCR and BCR as well as receptor tyrosine kinases, such as CSF1R and FLT3. Together, these studies suggested that SLAP/SLAP2 adaptor function has a role in the recruitment of CBL to activated tyrosine kinase complexes, facilitating phosphorylation and proximity to substrates. Our results reveal a more complex role for SLAP/SLAP2 in which the C-terminal domain binding to CBL also regulates ubiquitin ligase activity. Developmentally regulated and cell type specific expression of SLAP/SLAP2 therefore, would modulate CBL activity in specific cellular contexts.

SLAP activation of CBL could allow the ubiquitination of substrates in the absence of full CBL activation by tyrosine phosphorylation. Such a mechanism could contribute to the constitutive ubiquitination of TCR CD3⊐ chain in double positive (DP) thymocytes during T-cell development. A prior study demonstrated that the MHC-independent tonic ubiqutination of the TCR:CD3 complex requires both CBL and SLAP expression and coincides with the timing of upregulated SLAP expression in DP thymocytes. In this context, SLAP binding may serve to promote ubiquitination of substrates in the absence of activated TK signaling and CBL tyrosine phosphorylation by favoring the catalytically competent conformation. Alternatively, in the presence of an activated tyrosine kinase, SLAP2 binding and displacement of the TKBD bound LHR might facilitate Y371 phosphorylation and lower the threshold for CBL activation.

In addition to the role of the SLAP/SLAP2 carboxy-terminal tail in activating CBL ubiquitin ligase activity, SLAP/SLAP2 binding induced conformational changes in CBL may also influence substrate selection, as well as protein-protein interactions related to CBL adaptor function. Although we did not observe any differences in ubiquitin chain linkages assembled by phosphorylated CBL or SLAP bound CBL in vitro, it is possible that distinct mechanisms of activation could influence substrate ubiquitination qualitatively, resulting in distinct protein fates. For example, SLAP/SLAP2 binding might allow ubiquitination of substrates bound to CBL in regions distinct from TKBD pY-substrate binding pocket. Indeed, our data indicate that SLAP2 is ubiquitinated by CBL in vitro. Together, our observations suggest that SLAP/SLAP2 binding to CBL could provide a mechanism to tune CBL activity or substrate selection in the context of different degrees of tyrosine kinase signaling.

In conclusion, our results reveal a novel phosphorylation independent protein-protein interaction interface on the CBL TKBD and demonstrate that SLAP/SLAP2 adaptor protein binding to this site is an additional mechanism to regulate CBL ubiquitin ligase activity. Furthermore, the presence of SH2 and SH3 domains as well as a myristoylation modification support a model in which SLAP/SLAP2 binding regulates multiple context specific aspects of CBL function.

Coordinates and structure factors have been deposited in the Protein Data Bank with accession number 6XAR.

### Methods

### Cloning, Strains, Protein Overexpression and Purification

Residues 28-259 of mouse SLAP2, 29-261 of human SLAP2, or 19-254 of mouse SLAP were cloned in frame into a modified pET32a (pET32a-mod) vector (with a TEV cleavage site downstream of the His tag and 17 residues upstream of the thrombin cleavage site; gift from Gil Prive lab, UHN) with BamHl/Bglll and Xhol sites, to express thioredoxin (Trx)-His(6)-mSLAP2, -hSLAP2, and -mSLAP. Using standard QuikChange methods, 19 residues downstream of the TEV cleavage site were deleted from Trx-His(6)-hSLAP2, thus abolishing the thrombin cleavage site and placing the TEV cleavage site in closer proximity to the protein N-terminus, to generate Trx-His(6)-Δlinker-hSLAP2. Trx-His(6)-hSLAP2, Trx-His(6)-Δlinker-hSLAP2, and Trx-His(6)-mSLAP were cloned in frame into multiple cloning site (MCS) 1 of pETDuet-1 (Novagen), with CBL (47-357) cloned in frame into MCS2 of the same pETDuet-1 vector, for coexpression of SLAP2 or SLAP and CBL (Duet-His-hSLAP2-CBL, Duet-His-Δlinker-hSLAP2-CBL, and Duet-His-mSLAP-CBL). Deletion of residues 198-229 and 255-261 from SLAP2 in Duet-His-hSLAP2-CBL and point mutation of residues within Duet-His-Δlinker-hSLAP2-CBL were generated by standard QuikChange site-directed mutagenesis methods. Residues 25-357 and 2-436 of CBL were cloned in frame into pGEX4T-1 to express glutathione-S-transferase (GST)-CBL and GST-CBL TKBD LHR RING. Point mutations were generated in Trx-His(6)-hSLAP2 and GST-CBL TKBD LHR RING as above. GST-EphA4 (591-896) plasmid was a gift from Frank Sicheri (Lunenfeld-Tanenbaum Research Institute).

For purification of recombinant SLAP2 for crystallization trials and in vitro phosphorylation experiments, Trx-His(6)-mSLAP2 was transformed into E. coli BL-21 cells and grown in 8 L Luria Bertani (LB) media supplemented with 50 µg/ml ampicillin (Amp) overnight at 16° C. (A₆₀₀ = 0.6-0.9 and 0.5 mM isopropyl-β-D-thiogalactopyranoside (IPTG) induction). Cells were collected by centrifugation and cell pellet frozen at -80° C. Cell pellet was thawed and resuspended in ~150 mL high-salt lysis buffer (50 mM HEPES pH 7.5, 0.5 M NaCl, 10% glycerol, 10 mM imidazole, 10 mM β-mercaptoethanol (βME), 10 mM MgCl₂, 5 mM CaCl₂, cOmplete EDTA-free protease inhibitor cocktail tablets (inhibitor tablets) (Roche Applied Science), benzonase nuclease, 1 mM phenylmethylsulfonyl fluoride (PMSF)) and lysed by three cycles of high pressure homogenization (Emulsiflex) and two cycles of sonication on ice. Following centrifugation, supernatant was mixed with Ni-NTA agarose (Qiagen) for 90 min by gently nutating at 4° C. Resin was washed with 6 × 25 mL of wash buffer (50 mM HEPES pH 7.5, 0.5 M NaCl, 10% glycerol, 20 mM imidazole, 10 mM βME, 10 mM MgCl₂, 5 mM CaCl₂) and SLAP2 protein eluted with wash buffer containing increasing concentrations of imidazole (75, 150, 225, 300 mM imidazole, 28 mL total elution volume). The Trx-His(6) tag was cleaved by addition of 300 units of thrombin (Sigma T4648) directly to the eluate and the solution dialyzed (Slide-A-Lyzer dialysis cassettes, Thermo Scientific, 3500 MWCO) overnight at room temperature (rt) against 2 L dialysis buffer (25 mM Hepes pH 7.5, 0.4 M NaCl, 4% glycerol, 10 mM imidazole, 5 mM βME, 5 mM MgCl₂, 5 mM CaCl₂). The solution was removed from the dialysis cassette, centrifuged at 4000 rpm for 7 min to remove precipitate, and PMSF added to the soluble portion to 1 mM. This SLAP2 solution was passed very slowly over the same aliquot of Ni-NTA resin (washed since elution) to remove Trx-His(6), concentrated with a centrifugal filter unit (Amicon Ultra, Millipore), and flash frozen in liquid nitrogen for storage at -80° C.

For purification of recombinant CBL for crystallization trials, GST-CBL was overexpressed and cells harvested as above (except 6.5 hours at 37° C. or overnight at 30° C. and 1 mM IPTG induction). Cell pellet was resuspended in ~100 mL lysis buffer (50 mM HEPES pH 7.5, 0.5 M NaCl, 10% glycerol, 10 mM βME, 2 mM MgSO₄, inhibitor tablets, benzonase nuclease), and lysed as above. Following centrifugation, supernatant was mixed with Glutathione Sepharose 4B (GE Healthcare) for 90 min by nutating at 4° C. Resin was washed with 6 × 25 mL of wash buffer (50 mM HEPES pH 7.5, 0.5 M NaCl, 10% glycerol, 10 mM βME, 5 mM CaCl₂) and 150 units of thrombin (Sigma #T4648) added directly to the resin overnight at rt. CBL protein was eluted by washing resin with elution buffer (25 mM HEPES pH 7.5, 0.4 M NaCl, 5% glycerol, 5 mM βME), concentrated as above, and stored stably at 4° C. for weeks.

Approximately 33 mg of CBL or 10 mg of mSLAP2 were loaded individually onto a HiLoad 26/60 Superdex 75 gel filtration column (GE Healthcare Life Sciences) equilibrated with GF buffer (25 mM HEPES pH 7.5, 0.4 M NaCl, 2% glycerol, 5 mM βME) and protein elution detected by monitoring A₂₈₀. Fractions corresponding to isolated CBL or SLAP2 protein were assessed for purity by 12.5% SDS-PAGE analysis. Purified CBL and SLAP2 protein were mixed in a 1:1 molar ratio, concentrated to 1 mL, and loaded onto the same gel filtration column as above. Fractions corresponding to co-elution of CBL and SLAP2 were assessed for purity by 12.5% SDS-PAGE analysis and concentrated to 11.2 mg/mL. (Concentration was estimated by measuring the absorbance of the protein at 280 nm and calculating its concentration using a combined CBL/SLAP2 molecular extinction coefficient of 94810 M⁻¹ cm⁻¹ as predicted by ProtParam (http://web.expasy.org).

For co-purification of CBL and SLAP2/SLAP, Duet-His-hSLAP2-CBL 29-261 Δ198-229 and 29-254 Δ198-229, Duet-His-Δlinker-hSLAP2-CBL WT and point mutants, and Duet-His-mSLAP-CBL WT and point mutants were overexpressed in E. coli BL-21 as above or TKB1 competent cells (Stratagene) as per manufacturer’s instructions, and cells harvested as above (except 100 mL of LB, overnight at 16-18° C., and 0.35-0.5 mM IPTG induction). Cell pellets were resuspended in either high or low salt (as for high salt except 25 mM HEPEs pH 7.5, 150 mM NaCl) lysis buffer and purified as above on Ni-NTA agarose (Qiagen) with wash buffer (25 mM HEPEs pH 7.5, 150 mM NaCl, 2% glycerol, 20 mM imidazole, 10 mM βME). Bead slurry was mixed with SDS 2X loading buffer (125 mM Tris pH 6.8, 4% SDS, 20% glycerol, 0.715 M βME, bromophenol blue), boiled, and analyzed by 10 or 12.5% SDS-PAGE, stained with Coomassie blue.

For purification of SLAP2, pSLAP2, CBL-TKBD-LHR-RING (CBL) and pCBL for ubiquitination assays, GST-CBL-TKBD-LHR-RING and Trx-His(6)-hSLAP2 WT and point mutants were transformed into E. coli BL-21 as above or TKB1 competent cells (Stratagene) as per manufacturer’s instructions. 100 mL cultures were grown overnight at 18° C. (A₆₀₀ = 0.6-0.9 and 0.4 mM IPTG induction). TKB1 cultures were pelleted, resuspended in TK induction media, and tyrosine kinase expression induced as per manufacturer’s instructions. Cultures were pelleted at 4° C. and stored at -80° C. CBL pellets were resuspended in 3 ml each of NP-40 lysis buffer (50 mM HEPES pH 7.5, 150 mM NaCl, 10% glycerol, 1% NP-40, 10 mM NaF, 1.5 mM MgCl₂, 1 mM EDTA pH 8.0, 1 mM Na₃VO₄, and inhibitor tablets) and processed as above. CBL and pCBL were purified using glutathione sepharose 4B resin as above, followed by three washes with NP-40 wash buffer (20 mM HEPES pH 7.5, 150 mM NaCl, 5% glycerol, 0.1% NP-40, 5 mM βME, 0.5 PMSF, 0.5 mM Na₃VO₄), three washes with PBS wash buffer (PBS pH 7.4, 5 mM βME, 0.5 mM Na₃VO₄), and incubation with thrombin protease (Sigma #T4648). SLAP2 pellets were resuspended in 3 mL each of lysis buffer (50 mM HEPES pH 7.5, 0.5 M NaCl, 10% glycerol, 1% NP-40, 10 mM imidazole, 10 mM NaF, 5 mM βME, 5 mM caproic acid, 1.5 mM MgCl₂, 1 mM Na₃VO₄, benzonase nuclease (Novagen #70746), and inhibitor tablets) and processed as above. SLAP2 and pSLAP2 were isolated using Ni-NTA agarose resin as above, followed by three washes with 50 mM HEPES pH 7.5, 0.5 M NaCl, 2% glycerol, 20 mM imidazole, 5 mM βME, 0.5 mM Na₃VO₄, three washes with PBS wash buffer, and incubation with TEV protease. Protein concentrations were determined through Bradford assays.

For purification of GST-EphA4 for in vitro phosphorylation experiments, GST-EphA4 was overexpressed and cells harvested as above (except 2 L of LB, overnight at rt, and 0.15 mM IPTG induction). Cell pellets were resuspended in lysis buffer (50 mM HEPES pH 7.5, 0.5 M NaCl, 10% glycerol, 3 mM DTT, 1 mM MgSO₄, inhibitor tablets, benzonase nuclease) and prepared as above for GST-CBL. Resin was washed with 4 × 25 mL of PBS, pH 7.4, 3 mM DTT.

### Crystallization and Structure Determination

Co-eluted CBL/SLAP2 (5 mg/mL) was mixed in a hanging drop with equal volume (200 nL) of 0.1 M Hepes pH 7.5, 10% (w/v) PEG8000 at rt using a Mosquito robot (TTP Labtech). Small rod-like crystals were observed after approximately five months. A solution of 50% glycerol was added to the drop prior to harvesting and flash freezing the crystals in liquid nitrogen. Diffraction data was collected at the Advanced Photon Source and processed with Mosflm and QuickScale (Pointless, Aimless/Scala, Ctruncate) software to 2.5 Å. Molecular replacement was performed with Phenix software (Phaser_MR) using CBL TKBD (PDB id: 1B47) as a model. Anisotropic scaling of the data (services.mbi.ucla.edu/anisoscale) showed strong anisotropy (25.99 Å²) and determined limits of 2.5, 2.8, 2.5 Å. After multiple iterative cycles of model building in Coot and refinement with Phenix_refine, the Rfree was 33.0%. A Feature Enhanced Map (FEM) was calculated using Phenix software and polyGlycine chains placed in the unassigned density using the “Find Helices and Strands” feature in Phenix, the new FEM, and the improved model. Poly-glycines were manually converted to poly-alanines, and then to mSLAP2 residues ²⁴⁰GLRESLSSYISLAEDP²⁵⁵ in both chains of unassigned density. Real space refinement in Coot placed the SLAP2 residues perfectly within the previously unassigned density, and refinement in Phenix reduced the Rfree to 31.2%. Using the FEM, residues ²³⁷LSE²³⁹ were assigned, as well as water molecules in the next round of refinement, reducing Rfree to 30.4%. Final rounds of refinement reduced Rfree to 29.3% (Rfactor 24.9%). Symmetry related molecules, distances, superpositions, and root mean square deviations were calculated in Coot. Accessible surface area was calculated with Areaimol from the CCP4 program suite.

### In Vitro Phosphorylation and Detection by Mass Spectrometry

Purified mSLAP2 was incubated with GST-EphA4, 50 mM Hepes pH 7.5, 20 mM MgCl₂, 5 mM adenosine triphosphate (ATP), 5 mM DTT, and 1 mM Na₃VO₃ overnight at rt. Reaction was boiled with SDS 2X loading buffer, loaded on 12.5% SDS-PAGE gel, and transferred to polyvinylidene difluoride (PVDF) membrane. PVDF membrane was immunoblotted with anti-pTyr antibody 4G10 (Upstate Biotechnology, Inc.) as per manufacturer’s protocol. A second kinase reaction was boiled with SDS 2X loading buffer and run in multiple lanes of a 15% SDS-PAGE gel. The gel was stained with Coomassie and the upper band observed in the presence of EphA4 excised from the gel. Gel bands were treated and digested with either trypsin or GluC protease as per SPARC BioCentre’s in-gel digestion protocol (https://lab.research.sickkids.ca/sparc-molecular-analysis/services/mass-spectrometry/mass-spectrometry-sample-protocols/). Digested peptides were subjected to LC-MS/MS (60 min gradient, Thermo LTQ Orbitrap) and the raw data searched with PEAKS software against the mouse proteome, with carbamidomethylation as a fixed modification and deamidation (NQ), oxidation (M), and phosphorylation (STY) as variable modifications.

### In Vitro Autoubiquitination Assays

In vitro ubiquitination reactions were prepared with 20 pmol of CBL or pCBL and 60 pmol of hSLAP2 or phosphorylated hSLAP2 (pSLAP2) for both WT and mutants. Reagents were thawed and reactions prepared on ice. CBL and SLAP2 were mixed in PBS (with 5 mM βME, 5 mM caproic acid, 0.5 mM PMSF, and 0.5 mM Na₃VO₄) at rt for 5-10 min before addition of master mix (42 pmol E1 (UBE1, Boston Biochem or Ubiquitin-Proteasome Biotechnologies), 0.1 µmol E2 (UbcH5b/UBE2D2, Boston Biochem #E2-622), 1 mmol ATP, and 11.6 µmol ubiquitin (Boston Biochem #U-100H) in reaction buffer (0.2 M Tris pH 7.5, 10 mM MgCl₂, 2 mM DTT). Reactions were incubated at 30° C. shaking at 500-700 rpm for 60-110 minutes and terminated by addition of SDS 2X loading buffer and boiling for 5-10 minutes. Equal volumes of each reaction were loaded on 12.5% SDS-PAGE gel, transferred to PVDF membrane, and immunoblotted for ubiquitin as per standard protocols (anti-ubiquitin antibody P4G7, BioLegend #838701, 1:1000; anti-mouse IgG HRP-linked antibody, Cell Signaling Technology #7076, 1:10000; Western Lightning Plus ECL, Perkin Elmer).

Ubiquitination reactions were set up as above (25 µL total volume) in triplicate and diluted with reaction buffer after completion (1:40 for pCBL, 1:15 for all other reactions). Each reaction triplicate was added in triplicate to individual wells of a 384-well E3LITE Customizable Ubiquitin Ligase Kit (Life Sensors) plate, prewashed three times with PBS, and incubated for 60 minutes at rt on a rotating platform to capture ubiquitinated proteins. Wells were washed three times with PBS plus 0.1% Tween (PBS-T) and 25 uL of detection solution 1 (1:1000 dilution in PBS-T + 5% BSA) added to each well. After 50 min incubation, the wells were washed three times with PBS-T, 25 µL of streptavidin peroxidase polymer ultrasensitive antibody (Sigma #S2438, 1:10000 dilution in PBS-T, 5% BSA) added to each well, and the plate incubated for 50 min. Wells were washed four times with PBS-T and 25 µL of prepared Immobilon Western Chemiluminescent HRP Substrate (Millipore #P90719) added to each well immediately prior to the plate being read on a Synergy Neo2 Plate Reader (BioTek Instruments). The average luminescent signal for each condition was divided by that of CBL WT to determine fold change over CBL. Standard deviation of the reaction averages was calculated and used as a measure of error. Each reaction set was analyzed at least twice with fresh protein purifications.

### Chromatography-Mass Spectrometry Analysis of Ubiquitination Reactions

Ubiquitin linkages were monitored by a semi-quantitative mass spectrometry method that detects diglycine-modified ubiquitin “linkage reporter” peptides. In vitro autoubiquitination reactions were prepared as above, lyophilized, re-suspended in 9 M urea, 50 mM ammonium bicarbonate, 5 mM DTT, and incubated for 20 min at 60° C. Samples were cooled to rt, treated with 10 mM iodoacetamide for 30 min at rt in the dark, diluted with 50 mM ammonium bicarbonate to reduce urea to ~2 M, and treated with sequencing-grade, TPCK-treated, modified trypsin (Promega) for 16 hrs at 37° C. The resulting peptide samples were desalted using C18 chromatography columns and lyophilized. Peptides were re-suspended in 0.1% formic acid and analyzed by LC-MS/MS. LC was conducted using a C18 pre-column (Acclaim PepMap 100, 2 cm x 75 µm ID, Thermo Scientific) and a C18 analytical column (Acclaim PepMap RSLC, 50 cm x 75 µm ID, Thermo Scientific), running a 120 min reversed-phase gradient (0-40% ACN in 0.1% formic acid) at 225 nl/min on an EASY-nLC1200 pump (Proxeon) in-line with a Q-Exactive HF mass spectrometer (Thermo Scientific). A MS scan was performed with a resolution of 60,000 followed by up to 20 MS/MS scans (minimum ion count of 1000 for activation) using higher energy collision induced dissociation (HCD) fragmentation. Dynamic exclusion was set for 5 seconds (10 ppm; exclusion list size = 500). For peptide and protein identification, Thermo .RAW files were converted to .mzML format using ProteoWizard (v3.0.10800), then searched using X!Tandem (X!TANDEM Jackhammer TPP v2013.06.15.1) and Comet (v2014.02 rev.2) against the human RefSeq v45 database (containing 36113 entries). Search parameters specified a parent ion mass tolerance of 10 ppm and a MS/MS fragment ion tolerance of 0.4 Da, with up to two missed cleavages allowed for trypsin (excluding K/RP). Variable modifications of 0.984013 on N, 0.984014 on Q, 15.99491 on M, 114.04293 on K, 79.96633 on Y, 79.966329 on S, 79.96633 on T and 57.021459 on C were allowed. Data were filtered through the TPP (v4.7 POLAR VORTEX rev 1) with general parameters set as -p0.05 -x20 -PPM. For label-free MS1-level quantification, raw files were analyzed using MaxQuant (v1.6).

### Ubiquitination and Substrate Binding in Cells

COS-7 cells were serum starved for 24 hours (~80% confluent), followed by a 15 minute stimulation with 100 ng/mL EGF. Cells were lysed in 200 µL PLC lysis buffer/plate (50 mM HEPES pH 7.5, 150 mM NaCl, 1.5 mM MgCl₂, 1 mM EGTA pH 8.0, 10% glycerol, 1% Triton X-100, supplemented with 10 mM NaF, Na₃VO₄, cOmplete EDTA-protease inhibitor tablet (Roche)) and protein concentration determined by Bradford assay. Lysate (500 µg) was incubated for 2 hours at 4° C. with the equivalent of 30 µg GST-CBL-TKBD-LHR-RING WT or mutants captured on Glutathione Sepharose 4B as above. Samples were prepared for SDS-PAGE and immunoblot as described above, with anti-EGFR antibody (D38B1; Cell Signaling Tech).

HEK 293T cells were transfected using Lipofectamine 2000 (ThermoFisher), as per manufacturer’s instructions and standard procedures, with 3 µg HA-tagged full length CBL (in pCDEF3), 1 µg FLAG-tagged EGFR (in pcDNA 3.1), and/or 3 µg His-Ubiquitin (octameric ubiquitin construct with N-terminal His₆ tag for each), as indicated. Promoter matched backbone DNA was used where necessary to give 7 µg of DNA per plate for all conditions. After 24 hours, cells were harvested in cold PBS, pelleted by centrifugation, lysed in cold PLC lysis buffer, and protein concentration measured by Bradford assay. For lysate immunoblots, 50 µg samples were prepared in 2X SDS loading buffer and boiled for 10 min. For immunoprecipitation, 1 mg of lysate was incubated with pre-washed anti-FLAG M2 affinity gel (Sigma-Aldrich, #A2220) for 2 hours at 4° C. with gentle nutation. Resin was pelleted at 5000 rcf for 30 seconds at 4° C. and washed three times with lysis buffer. After the last wash, resin was mixed with 70 µL of 2X SDS loading buffer and boiled for 10 minutes. For immunoblotting, immunoprecipitation samples were divided between two gels. Lysate and immunoprecipitation samples were resolved by 8% SDS-PAGE and immunoblotted using standard protocols as described above. Where indicated, the following primary antibodies were used: 1 µg/mL anti-FLAG M2 (Sigma-Aldrich #F1804), 0.2 µg/mL anti-HA F-7 (Santa Cruz Biotechnology #sc7392), 0.5 µg/mL anti-His₆ (Roche #11 922 416 001). Images were acquired using a BioRad ChemiDoc imager. Figures are representative of four biological replicates.

### Example 2: High-Throughput Screen for CBL Ubiquitin Ligase Activators

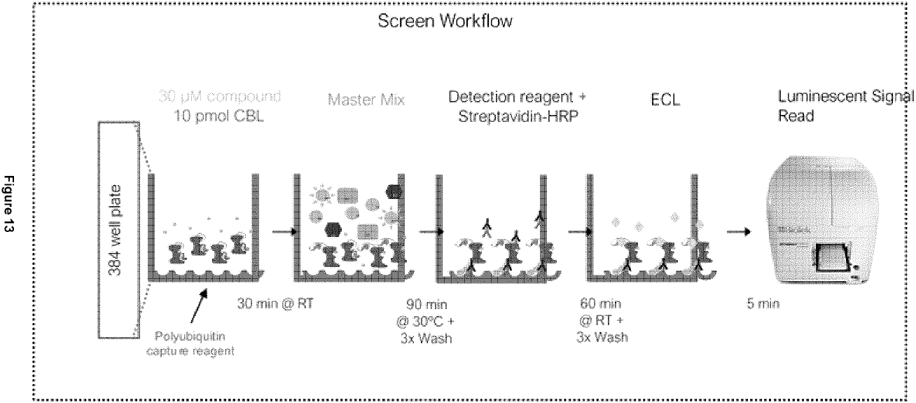

As an approach to identify novel compounds that mimic SLAP2 binding, and thus stimulate CBL E3 ligase activity, an in vitro autoubiquitination assay was developed based on a modification of a commercially available ELISA based assay (LifeSensors, Inc) and adapted to a 384 well high throughput screen (HTS) format (FIG. 13). The assay uses purified recombinant CBL protein containing the TKBD-LHR-RING region and has been optimized using reference controls; phosphorylated CBL, and CBL with or without addition of purified pSLAP2. Reaction components contained in a master mix (E1, E2, free Ub), test compound and purified CBL are free in solution allowing native protein interactions, and polyubiquitin chains are captured by an immobilized ubiquitin binding domain and quantified using an anti-ubiquitin antibody detection method.

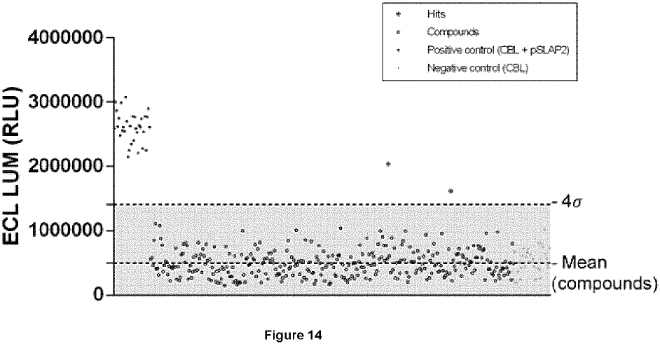

Peptidomimetic Screen: A library of 3000 α-helix and β/γ-turn peptidomimetic compounds were screened across 10 384 well plates. A primary screen yielded 14 hits from the OICR Peptidomimetic library (0.47% hit rate). A hit was defined as any compound producing a luminescent signal at least 4 Standard Deviations from the mean of all compounds on the plate (FIG. 14).

Validation experiment: Triplicate wells of hit compounds were randomly dispersed on plates and hits were confirmed based on a percent activation cutoff of at least 20% in at least 2 wells.

Confirmed Hits: 10/14 hits were confirmed from the Peptidomimetic library as being active:
1. N-[(2-chloro-6-fluorophenyl)methyl]-8-methyl-3,4-dihydro-2H-1,5-benzodioxepine-7-carboxamide;
2. N-(3-bromophenyl)-5-(5-cyclobutyl-1,3,4-oxadiazol-2-yl)thiophene-2-sulfonamide;
3.3-chloro-N-{3-cyclobutyl-[1,2,4]triazolo[4, 3-a]pyrid in-8-yl}-4-methoxybenzene-1-sulfonamide;
4. N-[(4-methoxyphenyl)methyl]-3-methyl-1-[3-(trifluoromethyl)benzenesulfonyl]piperidine-3-carboxamide;
5. N-[4-chloro-3-(trifluoromethyl)phenyl]-4-(5-cyclobutyl-1,2,4-oxadiazol-3-yl)thiophene-2-sulfonamide;
6. 5-(2-cyclobutyl-1,3-oxazol-5-yl)-N-[4-(trifluoromethoxy)phenyl]thiophene-2-sulfonamide;
7. 1-(4-methylbenzenesulfonyl)-N-(naphthalen-1-yl)-5-oxopyrrolidine-2-carboxamide;
8. 2-{4-[(4-methylphenyl)methyl]-2,3-dioxopiperazin-1-yl}-N-[4-(propan-2-yl)phenyl]acetamide;
9. N-(2-{4-[4-(5-cyclobutyl-1,2,4-oxadiazol-3-yl)phenyl]piperazin-1-yl}-2-oxoethyl)furan-2-carboxamide;
10. 5-oxo-3-phenyl-N-[3-(propan-2-yloxy)propyl]-5H-[1,3]thiazolo[3,2-a]pyrimidine-6-carboxamide.

1. An agent that inhibits CBL autoinhibition.

2. The agent of claim 1, wherein the agent acts independently of phosphorylation.

3. The agent of claim 1, wherein the agent interacts with a tyrosine-kinase binding domain (TKBD), a linker helix region (LHR), or a RING domain of CBL.

4. The agent of claim 1, wherein the agent does not bind to the phospho-tyrosine binding site of the TKBD.

5. The agent of claim 1, wherein the agent interacts with a region of CBL that is distinct from the phospho-tyrosine binding site of the TKBD.

6. The agent of claim 1, wherein the agent interacts with the CBL regulatory cleft.

7. The agent of claim 6, wherein the CBL regulatory cleft is framed by the 4H bundle, the EF-hand, and the SH2 domain.

8. The agent of claim 7, wherein the CBL regulatory cleft is framed by helices αC and αD of the 4H bundle, helix αE2 and loop αE2-αF2 of the EF-hand, and helix αN, loop αN-βA, and strand βA of the SH2 domain.

9. The agent of claim 1, wherein the agent is a SLAP and/or SLAP2 mimetic.

10. The agent of claim 1, wherein the agent comprises or consists of a peptide, a polynucleotide, a small molecule, a lipid, a carbohydrate, or a combination thereof.

11. The agent of claim 10, wherein the peptide is an antibody or fragment thereof, a linear, cyclic, or branched peptide or a combination thereof, a glycopeptide, a fusion peptide, a stapled peptide, a peptidomimetic, or a combination thereof.

12. The agent of claim 11, wherein the peptide is linked to a small molecule, such as a drug, imaging, or targeting agent.

13. The agent of claim 10, wherein the polynucleotide comprises DNA and/or RNA.

14. The agent of claim 10, wherein the small molecule is a macrocyclic compound.

15. The agent of claim 1, wherein the agent is N-[(2-chloro-6-fluorophenyl)methyl]-8-methyl-3,4-dihydro-2H-1,5-benzodioxepine-7-carboxamide; N-(3-bromophenyl)-5-(5-cyclobutyl-1,3,4-oxadiazol-2-yl)thiophene-2-sulfonamide; 3-chloro-N-{3-cyclobutyl-[1,2,4]triazolo[4,3-a]pyridin-8-yl}-4-methoxybenzene-1-sulfonamide; N-[(4-methoxyphenyl)methyl]-3-methyl-1-[3-(trifluoromethyl)benzenesulfonyl]piperidine-3-carboxamide; N-[4-chloro-3-(trifluoromethyl)phenyl]-4-(5-cyclobutyl-1,2,4-oxadiazol-3-yl)thiophene-2-sulfonamide; 5-(2-cyclobutyl-1,3-oxazol-5-yl)-N-[4-(trifluoromethoxy)phenyl]thiophene-2-sulfonamide; 1-(4-methylbenzenesulfonyl)-N-(naphthalen-1-yl)-5-oxopyrrolidine-2-carboxamide; 2-{4-[(4-methylphenyl)methyl]-2,3-dioxopiperazin-1-yl}-N-[4-(propan-2-yl)phenyl]acetamide; N-(2-{4-[4-(5-cyclobutyl-1,2,4-oxadiazol-3-yl)phenyl]piperazin-1-yl}-2-oxoethyl)furan-2-carboxamide; and 5-oxo-3-phenyl-N-[3-(propan-2-yloxy)propyl]-5H-[1,3]thiazolo[3,2-a]pyrimidine-6-carboxamide; or any combination thereof.

16. The agent of claim 1, in a delivery system, such as a gene therapy platform, a liposome, a nanoparticle, a therapeutic cell treatment, or a combination thereof.

17. The agent of claim 1 for treatment of a disease or condition in which CBL inhibition or downregulation is implicated.

18. The agent of claim 17, wherein the disease or condition in which CBL inhibition or downregulation is implicated is cancer, moyamoya angiopathy, or Noonan syndrome.

19. The agent of claim 18, wherein the cancer is leukemia, lung cancer, or head and neck cancer.

20. The agent of claim 19, wherein the leukemia is AML, JMML, CMML, or CML.

21. The agent of claim 19, wherein the cancer is a cancer in which tyrosine kinase activity is implicated.

22. A SLAP and/or SLAP2 mimetic.

23. The mimetic of claim 22, wherein the mimetic comprises or consists of a peptide, a polynucleotide, a small molecule, a lipid, a carbohydrate, or a combination thereof.

24. The mimetic of claim 23, wherein the peptide is an antibody or fragment thereof, a linear, cyclic, or branched peptide or a combination thereof, a glycopeptide, a fusion peptide, a stapled peptide, a peptidomimetic, or a combination thereof.

25. The mimetic of claim 24, wherein the peptide is linked to a small molecule, such as a drug, imaging, or targeting agent.

26. The mimetic of claim 22, wherein the polynucleotide comprises DNA and/or RNA.

27. The mimetic of claim 22, wherein the small molecule is a macrocyclic compound.

28. The mimetic of claim 22, wherein the mimetic is N-[(2-chloro-6-fluorophenyl)methyl]-8-methyl-3,4-dihydro-2H-1,5-benzodioxepine-7-carboxamide; N-(3-bromophenyl)-5-(5-cyclobutyl-1,3,4-oxadiazol-2-yl)thiophene-2-sulfonamide; 3-chloro-N-{3-cyclobutyl-[1,2,4]triazolo[4,3-a]pyridin-8-yl}-4-methoxybenzene-1-sulfonamide; N-[(4-methoxyphenyl)methyl]-3-methyl-1-[3-(trifluoromethyl)benzenesulfonyl]piperidine-3-carboxamide; N-[4-chloro-3-(trifluoromethyl)phenyl]-4-(5-cyclobutyl-1,2,4-oxadiazol-3-yl)thiophene-2-sulfonamide; 5-(2-cyclobutyl-1,3-oxazol-5-yl)-N-[4-(trifluoromethoxy)phenyl]thiophene-2-sulfonamide; 1-(4-methylbenzenesulfonyl)-N-(naphthalen-1-yl)-5-oxopyrrolidine-2-carboxamide; 2-{4-[(4-methylphenyl)methyl]-2,3-dioxopiperazin-1-yl}-N-[4-(propan-2-yl)phenyl]acetamide; N-(2-{4-[4-(5-cyclobutyl-1,2,4-oxadiazol-3-yl)phenyl]piperazin-1-yl}-2-oxoethyl)furan-2-carboxamide; and 5-oxo-3-phenyl-N-[3-(propan-2-yloxy)propyl]-5H-[1,3]thiazolo[3,2-a]pyrimidine-6-carboxamide; or any combination thereof.

29. The mimetic of claim 22, in a delivery system, such as a gene therapy platform, a liposome, a nanoparticle, a therapeutic cell treatment, or a combination thereof.

30. The mimetic of claim 22 for treatment of a disease or condition in which CBL inhibition or downregulation is implicated.

31. The mimetic of claim 30, wherein the disease or condition in which CBL inhibition or downregulation is implicated is cancer, moyamoya angiopath, or Noonan syndrome.

32. The mimetic of claim 31, wherein the cancer is leukemia, lung cancer, or head and neck cancer.

33. The mimetic of claim 32, wherein the leukemia is AML, JMML, CMML, or CML.

34. The mimetic of claim 33, wherein the cancer is a cancer in which tyrosine kinase activity is implicated.

35. Recombinant SLAP and/or SLAP2 or a variant and/or fragment thereof that inhibits CBL autoinhibition.

36. The recombinant SLAP and/or SLAP2 or the active variant and/or fragment thereof of claim 33, wherein the variant comprises at least about 70%, 75%, 80%, 85%, 90%, 95%, 96%, 97%, 98%, 99%, or 99.5% sequence identity to:
MGNSMKSTPAPAERPLPNPEGLDSDFLAVLSDYPSPDISPPIFRRGEKLRVISDEGGWWK AISLSTGRESYIPGICVARVYHGWLFEGLGRDKAEELLQLPDTKVGSFMIRESETKKGFY SLSVRHRQVKHYRIFRLPNNWYYISPRLTFQCLEDLVNHYSEVADGLCCVLTTPCLTQST AAPAVRASSSPVTLRQKTVDWRRVSRLQEDPEGTENPLGVDESLFSYGLRESIASYLSLT SEDNTSFDRKKKSISLMYGGSKRKSSFFSSPPYFED (SLAP; SEQ ID NO:1) and/or
MGSLPSRRKSLPSPSLSSSVQGQGPVTMEAERSKATAVALGSFPAGGPAELSLRLGEPLTI VSEDGDWWTVLSEVSGREYNIPSVHVAKVSHGWLYEGLSREKAEELLLLPGNPGGAFLI RESQTRRGSYSLSVRLSRPASWDRIRHYRIHCLDNGWLYISPRLTFPSLQALVDHYSELA DDICCLLKEPCVLQRAGPLPGKDIPLPVTVQRTPLNWKELDSSLLFSEAATGEESLLSEGL RESLSFYISLNDEAVSLDDA (SLAP2; SEQ ID NO:2).

37. The recombinant SLAP and/or SLAP2 or the active variant and/or fragment thereof of claim 35, wherein the fragment comprises from about 3 to about 275 (SLAP) or 260 (SLAP2) amino acids, such as from about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 180, about 185, about 190, about 195, about 200, about 205, about 210, about 215, about 220, about 225, about 230, about 235, about 240, about 245, about 250, about 255, about 260, about 265, or about 270 amino acids to about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 180, about 185, about 190, about 195, about 200, about 205, about 210, about 215, about 220, about 225, about 230, about 235, about 240, about 245, about 250, about 255, about 260, about 265, about 270, or about 275 amino acids.

38. The recombinant SLAP and/or SLAP2 or the active variant and/or fragment thereof of claim 35, wherein the fragment comprises at least about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 180, about 185, about 190, about 195, about 200, about 205, about 210, about 215, about 220, about 225, about 230, about 235, about 240, about 245, about 250, about 255, about 260, about 265, or about 270 amino acids.

39. The recombinant SLAP and/or SLAP2 or the active variant and/or fragment thereof of claim 35, in the form of an antibody or fragment thereof, a linear, cyclic, or branched peptide or a combination thereof, a glycopeptide, a fusion peptide, a stapled peptide, a peptidomimetic, or a combination thereof.

40. The recombinant SLAP and/or SLAP2 or the active variant and/or fragment thereof of claim 39, wherein the peptide is linked to a small molecule, such as a drug, imaging, or targeting agent.

41. The recombinant SLAP and/or SLAP2 or the active variant and/or fragment thereof of claim 35 for treatment of a disease or condition in which CBL inhibition or downregulation is implicated.

42. The recombinant SLAP and/or SLAP2 or the active variant and/or fragment thereof of claim 41, wherein the disease or condition in which CBL inhibition or downregulation is implicated is cancer, moyamoya angiopath, or Noonan syndrome.

43. The recombinant SLAP and/or SLAP2 or the active variant and/or fragment thereof of claim 35, wherein the cancer is leukemia, lung cancer, or head and neck cancer.

44. The recombinant SLAP and/or SLAP2 or the active variant and/or fragment thereof of claim 43, wherein the leukemia is AML, JMML, CMML, or CML.

45. The recombinant SLAP and/or SLAP2 or the active variant and/or fragment thereof of claim 42, wherein the cancer is a cancer in which tyrosine kinase activity is implicated.

46. A fusion protein comprising:
the agent of claim 1, wherein the agent is a peptide;
a SLAP and/or SLAP2 mimetic, wherein the mimetic is a peptide; or a recombinant SLAP and/or SLAP2 or an active variant and/or fragment thereof that inhibits CBL autoinhibition;
fused to a second peptide.

47. The fusion protein of claim 46, wherein the second peptide is a therapeutic peptide, an imaging peptide, a targeting peptide, or a combination thereof.

48. A method of inhibiting CBL autoinhibition, the method comprising administering to a subject in need thereof an agent that inhibits CBL autoinhibition; a SLAP and/or SLAP2 mimetic; a recombinant SLAP and/or SLAP2 or an active variant and/or fragment thereof that inhibits CBL autoinhibition; or the fusion protein of claim 46.

49. The method of claim 48, for treating a disease or condition in which CBL inhibition or downregulation is implicated.

50. The method of claim 49, wherein the disease or condition in which CBL inhibition or downregulation is implicated is cancer, moyamoya angiopath, or Noonan syndrome.

51. The method of claim 50, wherein the cancer is leukemia, lung cancer, or head and neck cancer.

52. The method of claim 51, wherein the leukemia is AML, JMML, CMML, or CML.

53. The method of claim 50, wherein the cancer is a cancer in which tyrosine kinase activity is implicated.

54. Use of an agent that inhibits CBL autoinhibition a SLAP and/or SLAP2 mimetic; a recombinant SLAP and/or SLAP2 or an active variant and/or fragment thereof that inhibits CBL autoinhibition; or the fusion protein of claim 46 for inhibiting CBL autoinhibition.

55. The use of claim 54, for treatment of a disease or condition in which CBL inhibition or downregulation is implicated.

56. The use of claim 55, wherein the disease or condition in which CBL inhibition or downregulation is implicated is cancer, moyamoya angiopath, or Noonan syndrome.

57. The use of claim 56, wherein the cancer is leukemia, lung cancer, or head and neck cancer.

58. The use of claim 57, wherein the leukemia is AML, JMML, CMML, or CML.

59. The use of claim 56, wherein the cancer is a cancer in which tyrosine kinase activity is implicated.

60. A method of screening for an agent that inhibits CBL autoinhibition and/or an agent that is a SLAP and/or SLAP2 mimetic, the method comprising applying the agent to a composition comprising CBL and detecting a change in CBL activation, wherein an increase in CBL activation suggests that the agent inhibits CBL autoinhibition and/or is a SLAP and/or SLAP2 mimetic.

61. The method of claim 60, wherein CBL activation is determined by measuring assembly of polyubiquitin chains by CBL.

62. The method of claim 60, further comprising validating the agent.

63. The method of claim 62, wherein validating the agent comprises detecting ubiquitination in an immunoassay, such as an immunoblot, following application of the agent to CBL.

64. The method of claim 60, wherein the CBL comprises the TKBD-LHR-RING region.

65. The method of claim 60, wherein the CBL is recombinant.

66. The method of claim 60, wherein the method is a high-throughput method.

67. An agent identified by the method of claim 60.
